(19) United States
(12) Patent Application Publication
(10) Pub. No.: US 2005/0250184 A1
(43) Pub. Date: Nov. 10, 2005

(54) Peptide hormones zalpha48 and zsig97

(76) Inventors: Paul O. Sheppard, Granite Falls, WA (US); Brian A. Fox, Seattle, WA (US)

Correspondence Address: Michelle L. Lewis;ZymoGenetics, Inc., 1201 Eastlake Avenue East, Seattle, WA, 98102, US

(21) Appl. No.: 10/997,437

(22) Filed: Nov. 24, 2004

Related U.S. Application Data
(60) Provisional application No. 60/525,597, filed on Nov. 26, 2003.

(51) Int. Cl. A61K 38/22; C07K 14/575; C07K 16/26

(52) U.S. Cl. 435/69.4; 530/399; 435/320.1; 435/325; 514/12; 536/23.5; 530/388.24

(57) ABSTRACT

The present invention relates to polynucleotide and polypeptide molecules for zalpha48 and zsig97, two proteins that define a novel peptide hormone family. The DNA encoding the peptides and the encoded peptides are useful in treating disorders of the thyroid, prostate, neural dysfunction, immune dysfunction, diseases of the pancreas, adrenal gland, ovary, and pituitary. The present invention also includes methods for producing the protein, uses therefor and antibodies therefore.

This application claims the benefit of U.S. Provisional Application Ser. No. 60/525,597, filed Nov. 26, 2003, which is herein incorporated by reference.

### BACKGROUND OF THE INVENTION

Peptide hormones are a class of small proteins that are secreted into the bloodstream that often act to integrate the functions of the brain and other systems of the body. Peptide hormones have been shown to play a role in numerous biological functions including regulation of reproduction; growth; water and salt metabolism; temperature control; food and water intake; cardiovascular, gastrointestinal, and respiratory control; behavior; memory; and affective states; and nerve development and regeneration. Strand, F. L. Neuropeptides Regulators of Physiological Processes, MIT Press, 1999.

Peptide hormones may be divided into families with similar or identical genes that express large precursor molecules that encompass one or more active molecules. Often there is a strong evolutionary link between members of a peptide hormone family. For example, the pancreatic polypeptide family currently includes three members: peptide YY, pancreatic polypeptide, and neuropeptide Y. These three peptide hormones share aspects of structural organization at both the gene and processed peptide level, strongly suggesting a common genetic origin for these three peptides. However, the hormones show quite distinct tissue expression, with peptide YY expressed in the duodenum and exocrine pancreas, neuropeptide Y in neural tissue, and pancreatic polypeptide expressed only in the pancreatic islet cells. Krasinski et al., Ann. N.Y. Acad. Sci. 622: 73-88, 1990. Most peptide hormones are initially translated with a signal sequence that guides the molecule through the ribosome and into the rough endoplasmic reticulum of the cell. These molecules are known as preprohormones. Signalase is the endopeptidase that removes the signal sequences and produces a biologically inert prohormone. Only through further proteolysis will one or more active peptides be released from this molecule. For many peptide hormones, the prohormone step appears to be essential for the correct protein folding and disulfide formation of the final active peptide product. Final processing of the prohormone occurs in secretory granules including endoproteolysis, exoproteolysis, glycosylation, actylation and amidation, among other post-processing steps. Most peptide hormones are produced on demand, in response to specific regulatory signals.

Evidence exists that precursor polypeptides can be more effective upon administration than active protein alone. Polypeptide precursors of peptide hormones are therefore sought for the study of hormone-related physiological processes. Moreover, novel polypeptides and polypeptide precursors with peptide hormone functions are sought. Additionally, novel antagonists and agonists of newly discovered peptides are possible as well as synthetic analogs. The present invention provides such polypeptides for these and other uses that should be apparent to those skilled in the art from the teachings herein.

### DESCRIPTION OF THE INVENTION

The present invention addresses this need by providing novel polynucleotides, polypeptides and related compositions and methods.

Within one aspect the present invention provides an isolated polynucleotide comprising a sequence of amino acid residues that is at least 90% identical to the amino acid sequence as shown in SEQ ID NO: 2 from amino acid Asp 90 to amino acid Arg 133, from amino acid Asp 95 to amino acid Arg 133, from amino acid Cys 134 to amino acid Lys 151, from amino acids Gly 20 to amino acid Lys 151, and from amino acid Met 1 to amino acid Lys 151. It also provides a sequence of amino acid residues that is at least 90% identical to the amino acid sequence as shown in SEQ ID NO: 6 from Asp 69 to Asp 112, from Asp 74 to Asp 112, from Cys 113 to amino acid Thr 129, from amino acid Ser 21 to amino acid Thr 129, and from amino acid Met 1 to amino acid Thr 129. The invention also encompasses the isolated polypeptide sequences that encode the amino acid sequences described above.

Within a second aspect the present invention provides an expression vector comprising the following operably linked elements: a transcription promoter; a DNA segment encoding a polypeptide comprising an amino acid sequence as shown in SEQ ID NO: 2 or SEQ ID NO:6.

Within a third aspect the present invention provides a cultured cell into which has been introduced an expression vector according as disclosed above, wherein the cell expresses a polypeptide encoded by the DNA segment.

Within another aspect the present invention provides a DNA construct encoding a fusion protein, the DNA construct comprising: a first DNA segment encoding a polypeptide that is at least 90% identical to a sequence of amino acid residues selected from the group consisting of: the amino acid sequence as shown in SEQ ID NO: 2 from amino acid Asp 90 to amino acid Arg 133, from amino acid Asp 95 to amino acid Arg 133, from amino acid Cys 134 to amino acid Lys 151, from amino acids Gly 20 to amino acid Lys 151, and from amino acid Met 1 to amino acid Lys 151. The DNA construct can also comprise a first DNA segment encoding a polypeptide that is at least 90% to a sequence of amino acid residues selected from the group consisting of the amino acid sequence as shown in SEQ ID NO: 6 from Asp 69 to Asp 112, from Asp 74 to Asp 112, from Cys 113 to amino acid Thr 129, from amino acid Ser 21 to amino acid Thr 129, and from amino acid Met 1 to amino acid Thr 129

The DNA construct is connect to at least one other DNA segment encoding an additional polypeptide, wherein the first and other DNA segments are connected in-frame; and encode the fusion protein.

Within another aspect the present invention provides a fusion protein produced by a method comprising: culturing a host cell into which has been introduced a vector comprising the following operably linked elements: (a) a transcriptional promoter; (b) a DNA construct encoding a fusion protein as disclosed above; and (c) a transcriptional terminator; and recovering the protein encoded by the DNA segment.

Within another aspect the present invention provides a method of producing a polypeptide comprising: culturing a cell as disclosed above; and isolating the polypeptide produced by the cell.

Within another aspect the present invention provides a method of detecting, in a test sample, the presence of a modulator of zalpha48 or zsig97 protein activity, comprising: transfecting a zalpha48 or zsig97-responsive cell, with a reporter gene construct that is responsive to a zalpha48 or zsig97-stimulated cellular pathway; and producing a polypeptide by the method as disclosed above; and adding the polypeptide to the cell, in the presence and absence of a test sample; and comparing levels of response to the polypeptide, in the presence and absence of the test sample, by a biological or biochemical assay; and determining from the comparison, the presence of the modulator of zalpha48 or zsig97 activity in the test sample.

Within another aspect the present invention provides a method of producing an antibody to a polypeptide comprising the following steps in order: inoculating an animal with a polypeptide as discussed above, wherein the polypeptide elicits an immune response in the animal to produce the antibody; and isolating the antibody from the animal.

Within another aspect the present invention provides an antibody produced by the method as disclosed above, which binds to a polypeptide of SEQ ID NO: 2 or SEQ ID NO:6 as discussed above. In one embodiment, the antibody disclosed above is a monoclonal antibody.

Within another aspect the present invention provides a method for detecting pancreas, adrenal gland, ovary, or pituitary tissue in a patient sample, utilizing the zalpha48 protein comprising: obtaining a tissue or biological sample from a patient; incubating the tissue or biological sample with an antibody as disclosed above under conditions wherein the antibody binds to its complementary polypeptide in the tissue or biological sample; visualizing the antibody bound in the tissue or biological sample; and comparing levels and localization of antibody bound in the tissue or biological sample from the patient to a non-pancreas, adrenal gland, ovary, or pituitary control tissue or biological sample, wherein an increase in the level or localization of antibody bound to the patient tissue or biological sample relative to the non-pancreas, adrenal gland, ovary, or pituitary control tissue or biological sample is indicative of pancreas, adrenal gland, ovary or pituitary tissue in a patient sample. This method can also be used to detect thyroid or prostate tissue using the zsig97 protein.

Within another aspect the present invention provides a method for detecting pancreas, adrenal gland, ovary, or pituitary tissue in a patient sample utilizing the DNA sequence of zalpha48, comprising: obtaining a tissue or biological sample from a patient; labeling a polynucleotide comprising at least 14 contiguous nucleotides of SEQ ID NO:1 or the complement of SEQ ID NO:1; incubating the tissue or biological sample with under conditions wherein the polynucleotide will hybridize to complementary polynucleotide sequence; visualizing the labeled polynucleotide in the tissue or biological sample; and comparing the level and localization of labeled polynucleotide hybridization in the tissue or biological sample from the patient to a control non-pancreas, adrenal gland, ovary, or pituitary tissue or biological sample, wherein an increase in the level or localization of the labeled polynucleotide hybridization to the patient tissue or biological sample relative to the control non-pancreas, adrenal gland, ovary, or pituitary tissue or biological sample is indicative of pancreas, adrenal gland, ovary, or pituitary tissue in a patient sample. This method can also be used to detect thyroid or prostate tissue utilizing the DNA sequence of zsig97.

These and other aspects of the invention will become evident upon reference to the following detailed description of the invention.

Prior to setting forth the invention in detail, it may be helpful to the understanding thereof to define the following terms:

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), Glu-Glu affinity tag (Grussenmeyer et al., Proc. Natl. Acad. Sci. USA 82:7952-4, 1985), substance P, Flag™ peptide (Hopp et al., Biotechnology 6:1204-10, 1988), streptavidin binding peptide, or other antigenic epitope or binding domain. See, in general, Ford et al., Protein Expression and Purification 2: 95-107, 1991. DNAs encoding affinity tags are available from commercial suppliers (e.g., Pharmacia Biotech, Piscataway, N.J.).

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The terms “amino-terminal” (N-terminal) and “carboxyl-terminal” (C-terminal) are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of <10⁹ M-1.

The term “complements of a polynucleotide molecule” denotes a polynucleotide molecule having a complementary base sequence and reverse orientation as compared to a reference sequence. For example, the sequence 5′ ATGCACGGG 3′ is complementary to 5 CCCGTGCAT 3′.

The term “contig” denotes a polynucleotide that has a contiguous stretch of identical or complementary sequence to another polynucleotide. Contiguous sequences are said to “overlap” a given stretch of polynucleotide sequence either in their entirety or along a partial stretch of the polynucleotide. For example, representative contigs to the polynucleotide sequence 5′-ATGGCTTAGCTT-3′ are 5′-TAGCTTgagtct-3′ and 3′-gtcgacTACCGA-5′.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

The term “operably linked”, when referring to DNA segments, indicates that the segments are arranged so that they function in concert for their intended purposes, e.g., transcription initiates in the promoter and proceeds through the coding segment to the terminator.

The term “ortholog” denotes a polypeptide or protein obtained from one species that is the functional counterpart of a polypeptide or protein from a different species. Sequence differences among orthologs are the result of speciation.

“Paralogs” are distinct but structurally related proteins made by an organism. Paralogs are believed to arise through gene duplication. For example, α-globin, β-globin, and myoglobin are paralogs of each other.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired.

A “polypeptide” is a polymer of amino acid residues joined by peptide bonds, whether produced naturally or synthetically. Polypeptides of less than about 10 amino acid residues are commonly referred to as “peptides”.

“Probes and/or primers” as used herein can be RNA or DNA. DNA can be either cDNA or genomic DNA. Polynucleotide probes and primers are single or double-stranded DNA or RNA, generally synthetic oligonucleotides, but may be generated from cloned cDNA or genomic sequences or its complements. Analytical probes will generally be at least 20 nucleotides in length, although somewhat shorter probes (14-17 nucleotides) can be used. PCR primers are at least 5 nucleotides in length, preferably 15 or more nt, more preferably 20-30 nt. Short polynucleotides can be used when a small region of the gene is targeted for analysis. For gross analysis of genes, a polynucleotide probe may comprise an entire exon or more. Probes can be labeled to provide a detectable signal, such as with an enzyme, biotin, a radionuclide, fluorophore, chemiluminescer, paramagnetic particle and the like, which are commercially available from many sources, such as Molecular Probes, Inc., Eugene, Oreg., and Amersham Corp., Arlington Heights, Ill., using techniques that are well known in the art.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “receptor” denotes a cell-associated protein that binds to a bioactive molecule (i.e., a ligand) and mediates the effect of the ligand on the cell. Membrane-bound receptors are characterized by a multi-peptide structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

All references cited herein are incorporated by reference in their entirety.

The present invention is based in part upon the discovery of two proteins making up a novel family of peptide hormones, where each protein comprises peptide hormones that are produced upon post-translation proteolytic cleavage. The polypeptides have been designated zalpha48 and zsig97.

The novel peptide family of the present invention was initially identified through the homology of zalpha48 and zsig97 to each other, and the presence of peptide hormones within the protein coding region. Genomic DNA sequences were discovered and analyzed, including prediction of the exons that, for both genes, code for prepropeptides. Prohormone production was predicted by removal of the signal sequence. Analysis was also preformed to predict the proteolytic cleavage sites utilized to produce the mature peptide hormones.

The nucleotide sequence of human zalpha48 is described in SEQ ID NO:1, and its deduced amino acid sequence is described in SEQ ID NO:2. The nucleotide sequence of mouse zalpha48 is described in SEQ ID NO: 3 and its deduced amino acid sequence is described in SEQ ID NO 4. The nucleotide sequence of human zsig97 is described in SEQ ID NO:5 and its deduced amino acid sequence is described in SEQ ID NO: 6. Finally, the nucleotide sequence of mouse zsig97 is described in SEQ ID NO: 7 and its deduced amino acid sequence is described in SEQ IN NO:8. Comparison between these various amino acid sequences revealed that both zalpha48 and zsig97 have multiple dibasic cleavage sites, that produce peptides of predicted small size (5-40 kD), and lack of long hydrophobic segments, revealing these proteins produce small secreted molecules and form a new family of secreted peptide hormone molecules.

Analysis of the genomic DNA encoding the zalpha48 polypeptide revealed a cDNA (SEQ ID NO:1) containing an open reading frame encoding 151 amino acids (SEQ ID NO:2) comprising a human zalpha48 prepropeptide. The mouse ortholog was isolated and analyzed, revealing a cDNA sequence (SEQ ID NO: 3) with an open reading frame encoding 150 amino acids (SEQ ID NO: 4) comprising a mouse zalpha48 prepropeptide. Similarily, analysis of the genomic DNA encoding the zsig97 polypeptide revealed a cDNA (SEQ ID NO: 3) containing an open reading frame encoding 129 amino acids (SEQ ID NO: 4) comprising a human zsig97 prepropeptide. The mouse ortholog for this gene was isolated and analyzed, revealing a cDNA (SEQ ID NO: 7) containing an open reading frame encoding 127 amino acids (SEQ ID NO:8) comprising a mouse zsig97 prepropeptide. All of the prepropeptides have a putative cleavage site to remove the signal sequence and convert the protein into a propeptide, specifically at Gly 20 for both the mouse and human zalpha48 prepropeptide and at Ser 21 for the human and mouse zsig97 prepropeptide.

Table 1 discloses the amino acid cleavage sites of the various important structural aspects of zalpha48 and zsig97, including the signal sequence and mature peptides boundaries. Generally, cleavage occurs to the COOH side of the disclosed amino acid. As reference to the table makes clear, the present molecules produce three active peptides, mature one, mature two, and mature three. The mature one peptide has two alternative NH-cleavage sites, identified as #1 and #2 in the table. Thus, for human zalpha48 the mature one peptide is alternatively from amino acid Asp 90 to amino acid Arg 133 or from amino acid Asp 95 to amino acid Arg 133, while the mature one peptide for human zsig97 is alternatively from amino acid Asp 69 to amino acid Arg 112 or from amino acid Asp 74 to amino acid Arg 112. For human zalpha48 the mature two peptide is from amino acid Cys 134 to amino acid Lys 151 and for human zsig97 from amino acid Cys 113 to amino acid Thr 129. It should also be noted that a mature three peptide is also possible where there is only a cleavage one site in the peptides. Thus, for zalpha48, this mature three peptide would be amino acid Asp 90 to Lys 151 or amino acid Asp 95 to Lys 151. For zsig97 mature three peptide would be Asp 69 to Thr 129 or Asp 74 to Thr 129. Those skilled in the art will recognize that domain boundaries, exon endpoints, and cleavage sites are approximations based on sequence alignments, intron positions and splice sites, and may vary slightly; however, such estimates are generally accurate to within ±4 amino acid residues.

TABLE 1

| | | mature | | | | |
|---|---|---|---|---|---|---|
| | | one/ | mature | | | mature |
| | signal | three | one/ | mature | mature | two/ |
| | sequence | NH | three | one | two | three |
| Gene name | COOH | (#1) | NH (#2) | COOH | NH | COOH |
| Huzalpha48 | Gly 20 | Asp 90 | Asp 95 | Arg 133 | Cys 134 | Lys 151 |
| Muzalpha48 | Gly 20 | Asp 91 | Asp 96 | Arg 134 | Cys 135 | Glu 150 |
| Huzsig97 | Ser 21 | Asp 69 | Asp 74 | Arg 112 | Cys 113 | Thr 129 |
| Muzsig97 | Ser 21 | Asp 67 | Asp 72 | Arg 110 | Cys 111 | Thr 127 |

Further analysis of these molecules revealed a similar gene to protein structure, where the first exon of all the genes comprises the signal sequence, the second exon comprises a portion of the first half of the first mature peptide produced from this molecule. The second half of the mature one peptide (short three amino acids) is present in the third exon. The fourth exon comprises the last three amino acids of the mature one peptide as well as the full mature two peptide. This similarity in gene and protein structure argues for a common evolutionary source for these two genes and strongly supports the classification of these molecules into a new peptide hormone family. Table 2 discloses these exon endpoints as amino acids.

TABLE 2

| | Exon | Exon | Exon |
|---|---|---|---|
| Gene name | 1/2 | 2/3 | 3/4 |
| Huzalpha48 | Glu 81 | Leu 101 | Tyr 130 |
| Muzalpha48 | Glu 82 | Leu 102 | Tyr 131 |
| Huzsig97 | Ser 60 | Phe 80 | Tyr 109 |
| Muzsig97 | Ser 58 | Phe 78 | Tyr 107 |

An active zalpha48 or zsig97 polypeptide can be amidated in order to avoid further degradation from the COOH end of the mature peptides. It should also be noted that there are four conserved cysteine residues within the mature peptide regions, two occurring within the mature one peptide and two occurring within the mature two peptide. These cysteines are believed to be important to the formation of intra- and inter-disulfide bonds within and between the mature peptides. In addition to each active individual peptide molecule (i.e., mature one and mature two), an active polypeptide including both mature peptides can confer functional and biological properties of zalpha48 or zsig97 such as the preprohormone Met 1 to Lys 151 for zalpha48 or the preprohormone Met 1 to Thr 129 for zsig97. Moreover, the polypeptide from amino acid Gly 20 to Lys 151 of SEQ ID NO:2 can serve as a prohormone for zalpha48 and be post-translationally modified and cleaved into individual mature peptides, as can the polypeptide from amino acid Ser 21 to Thr 129 for zsig97.

The corresponding polynucleotides encoding the human zalpha48 polypeptide regions, domains, motifs, residues and sequences described above are as shown in SEQ ID NO:2, while those encoding the human zsig97 polypeptide regions, domains, motifs, residues, and sequences described above are as shown in SEQ ID NO 6.

The presence or absence of transmembrane regions, dibasic cleavage sites, cysteine residues, and conserved and low variance motifs generally correlates with or defines important structural regions in proteins. Regions of low variance (e.g., hydrophobic clusters) are generally present in regions of structural importance (Sheppard, P. et al., supra.). Such regions of low variance often contain rare or infrequent amino acids, such as Tryptophan. The regions flanking and between such conserved and low variance motifs may be more variable, but are often functionally significant because they relate to or define important structures and activities such as binding domains, biological and enzymatic activity, signal transduction, cell-cell interaction, tissue localization domains and the like.

The nucleotide sequences that encode the mature peptide one or mature peptide two, for example, can be used as a tool to identify new peptide hormone family members. For instance, reverse transcription-polymerase chain reaction (RT-PCR) can be used to amplify sequences encoding the mature peptides above from RNA obtained from a variety of tissue sources or cell lines. In particular, highly degenerate primers designed from the zalpha48 or zsig97 sequences are useful for this purpose.

Moreover the genomic structure of zalpha48 and zsig97 are readily determined by one of skill in the art by comparing the cDNA sequences and the translated amino acid sequences with the genomic DNA in which the gene is contained. For example, such analysis can be readily done using FASTA as described herein. As such, the intron and exon junctions in this region of genomic DNA have been determined for both the zalpha48 and zsig97 genes and have indicated a distinct pattern between the exons and the mature peptide coding regions, supporting a finding that these two genes are members of a novel peptide hormone family. Additionally, it has been determined that human zalpha48 maps to chromosome 2, specifically the 2p25.3 region and human zsig97 maps to chromosome 8, specifically 8q11.23.

The present invention is not limited to the expression of the sequence shown in SEQ ID NO:1 or SEQ ID NO:3. A number of truncated zalpha48 or zsig97 polynucleotides and polypeptides are provided by the present invention. These polypeptides can be produced by expressing polynucleotides encoding them in a variety of host cells. In many cases, the structure of the final polypeptide product will result from processing of the nascent polypeptide chain by the host cell, thus the final sequence of a zalpha48 or zsig97 polypeptide produced by a host cell will not always correspond to the full sequence encoded by the expressed polynucleotide. For example, expressing the complete zalpha48 or zsig97 sequence in a cultured mammalian cell is expected to result in removal of at least the secretory peptide, while the same polypeptide produced in a prokaryotic host would not be expected to be cleaved. By selecting particular combinations of polynucleotide and host cell, a variety of zalpha48 or zsig97 polypeptides can thus be produced. Differential processing of individual chains may result in heterogeneity of expressed polypeptides and the production of heterodimeric zalpha48 or zsig97 proteins. As such, the mature processed peptides, such mature one peptide and mature two peptide and as well as others disclosed herein, may be dimeric, or multimeric, and may be disulfide bonded through one or more of their conserved cysteines to form complexes of one or more polypeptides. For example, the two cysteine residues in mature one (shown at amino acids 111 and 125 in SEQ ID NO:2; amino acids 112 and 126 in SEQ ID NO:4;) would be candidate for disulfide bonding to an additional zalpha48 or zsig97 peptide, such as the Cys residue in repeat-1 (shown at amino acid 143 in SEQ ID NO:2), and the like. Table 3 discloses the amino acid locations of these conserved cysteines.

TABLE 3

| | Cysteine | Cysteine | Cysteine | Cysteine |
|---|---|---|---|---|
| | #1 | #2 | #1 | #2 |
| | mature | mature | mature | mature |
| Gene name | one | one | two | two |
| Huzalpha48 | 111 | 125 | 134 | 147 |
| Muzalpha48 | 112 | 126 | 135 | 148 |
| Huzsig97 | 90 | 104 | 113 | 126 |
| Muzsig97 | 88 | 102 | 111 | 124 |

One of skill in the art can readily determine, upon reference to Table 3, and the zalpha48 or zsig97 mature one or mature peptides as disclosed herein, the cysteine residues present in those fragments that can be disulfide bonded to form complexes of one or more polypeptides. One of skill in the art would also recognize that any combination of the zalpha48 or zsig97 cysteine-containing fragments disclosed herein could be disulfide bonded as dimers, and potentially multimers. In addition, zalpha48 or zsig97 polypeptides can be produced by other known methods, such as solid phase synthesis, methods for which are well known in the art. See, for example, Merrifield, J. Am. Chem. Soc. 85:2149, 1963; Stewart et al., Solid Phase Peptide Synthesis (2nd edition), Pierce Chemical Co., Rockford, Ill., 1984; Bayer and Rapp, Chem. Pept. Prot. 3:3, 1986; and Atherton et al., Solid Phase Peptide Synthesis: A Practical Approach, IRL Press, Oxford, 1989.

Northern blot analysis is expected to show that a transcript for zalpha48 is detected in glandular, neural, and reproductive tissues such as pancreas, adrenal gland, ovary, and pituitary. RT-PCR was performed to show where zalpha48 mRNA is expressed. The results showed that zalpha48 expression is tissue-specific, and evident in pancreas, adrenal gland, ovary, and pituitary but not other tissues examined. Additional analysis may reveal an zalpha48 transcript in more localized pancreatic, adrenal, ovary or pituitary tissue, specific cell types within those tissues, and in tumor cell lines. Such methods to determine such expression are well known in the art and disclosed herein.

Northern blot analysis is expected to show that a transcript for zsig97 is detected in glandular, neural, and immunological tissues such as thyroid, prostate, dendrites, and leukocytes such as monocytes. RT-PCR was performed to show where zsig97 mRNA is expressed. The results showed that zsig97 expression is tissue-specific, and evident in thyroid, prostate smooth muscle cell, prostate, KG-1 (a dendritic cell line), and THP-1 (as acute monocytic leukemia cell line) but not other tissues examined. Additional analysis may reveal a zsig97 transcript in more localized thyroid tissue, specific cell types within those tissues, and in other tumor cell lines. Such methods to determine such expression are well known in the art and disclosed herein.

The present invention also provides polynucleotide molecules, including DNA and RNA molecules that encode the zalpha48 and zsig97 polypeptides disclosed herein. Those skilled in the art will readily recognize that, in view of the degeneracy of the genetic code, considerable sequence variation is possible among these polynucleotide molecules. SEQ ID NO:9 is a degenerate DNA sequence that encompasses all DNAs that encode the zalpha48 polypeptide of SEQ ID NO:2. SEQ ID NO:10 is a degenerate DNA sequence that encompasses all DNAs that encode the zsig97 polypeptide of SEQ ID NO: 6. Those skilled in the art will recognize that the degenerate sequence of SEQ ID NO:9 and 10 also provides all RNA sequences encoding SEQ ID NO:2 and 6, respectively, by substituting U for T. Thus, zalpha48 polypeptide-encoding polynucleotides comprising nucleotide 1 to nucleotide 1258 of SEQ ID NO:1 as well as zsig97 polypeptide-encoding polynucleotides comprising nucleotide 1 to nucleotide 814, and their RNA equivalents are contemplated by the present invention. Table 4 sets forth the one-letter codes used within SEQ ID NOS: 9 and 10 to denote degenerate nucleotide positions. “Resolutions” are the nucleotides denoted by a code letter. “Complement” indicates the code for the complementary nucleotide(s). For example, the code Y denotes either C or T, and its complement R denotes A or G, A being complementary to T, and G being complementary to C.

TABLE 4

| | | | |
|---|---|---|---|
| Nucleotide | Resolution | Complement | Resolution |

| | | | |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NOS: 9 and 10, encompassing all possible codons for a given amino acid, are set forth in Table 5.

TABLE 5

One

| | | |
|---|---|---|
| Amino | Letter | Degenerate |

| | | | |
|---|---|---|---|
| Acid | Code | Codons | Codon |

| | | | |
|---|---|---|---|
| Cys | C | TGC, TGT | TGY |
| Ser | S | AGC, AGT, TCA, TCC, | WSN |
| | | TCG, TCT | |
| Thr | T | ACA, ACC, ACG, ACT | ACN |
| Pro | P | CCA, CCC, CCG, CCT | CCN |
| Ala | A | GCA, GCC, GCG, GCT | GCN |
| Gly | G | GGA, GGC, GGG, GGT | GGN |
| Asn | N | AAC, AAT | AAY |
| Asp | D | GAC, GAT | GAY |
| Glu | E | GAA, GAG | GAR |
| Gln | Q | CAA, CAG | CAR |
| His | H | CAC, CAT | CAY |
| Arg | R | AGA, AGG, CGA, CGC, | MGN |
| | | CGG, CGT | |
| Lys | K | AAA, AAG | AAR |
| Met | M | ATG | ATG |
| Ile | I | ATA, ATC, ATT | ATH |
| Leu | L | CTA, CTC, CTG, CTT, | YTN |
| | | TTA, TTG | |
| Val | V | GTA, GTC, GTG, GTT | GTN |
| Phe | F | TTC, TTT | TTY |
| Tyr | Y | TAC, TAT | TAY |
| Trp | W | TGG | TGG |
| Ter | • | TAA, TAG, TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequence of SEQ ID NOS: 2, 4, 6, and 8. Variant sequences can be readily tested for functionality as described herein.

One of ordinary skill in the art will also appreciate that different species can exhibit “preferential codon usage.” In general, see, Grantham, et al., Nuc. Acids Res. 8:1893-912, 1980; Haas, et al. Curr. Biol. 6:315-24, 1996; Wain-Hobson, et al., Gene 13:355-64, 1981; Grosjean and Fiers, Gene 18:199-209, 1982; Holm, Nuc. Acids Res. 14:3075-87, 1986; Ikemura, J. Mol. Biol. 158:573-97, 1982. As used herein, the term “preferential codon usage” or “preferential codons” is a term of art referring to protein translation codons that are most frequently used in cells of a certain species, thus favoring one or a few representatives of the possible codons encoding each amino acid (See Table 5). For example, the amino acid Threonine (Thr) may be encoded by ACA, ACC, ACG, or ACT, but in mammalian cells ACC is the most commonly used codon; in other species, for example, insect cells, yeast, viruses or bacteria, different Thr codons may be preferential. Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequence disclosed in SEQ ID NO:3 serves as a template for optimizing expression of polynucleotides in various cell types and species commonly used in the art and disclosed herein. Sequences containing preferential codons can be tested and optimized for expression in various species, and tested for functionality as disclosed herein.

Within preferred embodiments of the invention the isolated polynucleotides will hybridize to similar sized regions of SEQ ID NOS:1, 3, 5, or 7 or a sequence complementary thereto, under stringent conditions. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Numerous equations for calculating Tₘ are known in the art, and are specific for DNA, RNA and DNA-RNA hybrids and polynucleotide probe sequences of varying length (see, for example, Sambrook et al., Molecular Cloning: A Laboratory Manual, Second Edition (Cold Spring Harbor Press 1989); Ausubel et al., (eds.), Current Protocols in Molecular Biology (John Wiley and Sons, Inc. 1987); Berger and Kimmel (eds.), Guide to Molecular Cloning Techniques, (Academic Press, Inc. 1987); and Wetmur, Crit. Rev. Biochem. Mol. Biol. 26:227 (1990)). Sequence analysis software such as OLIGO 6.0 (LSR; Long Lake, Minn.) and Primer Premier 4.0 (Premier Biosoft International; Palo Alto, Calif.), as well as sites on the Internet, are available tools for analyzing a given sequence and calculating Tₘ based on user-defined criteria. Such programs can also analyze a given sequence under defined conditions and identify suitable probe sequences. Typically, hybridization of longer polynucleotide sequences, >50 base pairs, is performed at temperatures of about 20-25° C. below the calculated Tₘ. For smaller probes, <50 base pairs, hybridization is typically carried out at the Tₘ or 5-10° C. below. This allows for the maximum rate of hybridization for DNA-DNA and DNA-RNA hybrids. Higher degrees of stringency at lower temperatures can be achieved with the addition of formamide which reduces the Tₘ of the hybrid about 1° C. for each 1% formamide in the buffer solution. Suitable stringent hybridization conditions are equivalent to about a 5 h to overnight incubation at about 42° C. in a solution comprising: about 40-50% formamide, up to about 6×SSC, about 5× Denhardt's solution, zero up to about 10% dextran sulfate, and about 10-20 μg/ml denatured commercially-available carrier DNA. Generally, such stringent conditions include temperatures of 20-70° C. and a hybridization buffer containing up to 6×SSC and 0-50% formamide; hybridization is then followed by washing filters in up to about 2×SSC. For example, a suitable wash stringency is equivalent to 0.1×SSC to 2×SSC, 0.1% SDS, at 55° C. to 65° C. Different degrees of stringency can be used during hybridization and washing to achieve maximum specific binding to the target sequence. Typically, the washes following hybridization are performed at increasing degrees of stringency to remove non-hybridized polynucleotide probes from hybridized complexes. Stringent hybridization and wash conditions depend on the length of the probe, reflected in the Tm, hybridization and wash solutions used, and are routinely determined empirically by one of skill in the art.

As previously noted, the isolated polynucleotides of the present invention include DNA and RNA. Methods for preparing DNA and RNA are well known in the art. In general, RNA is isolated from a tissue or cell that produces large amounts of zalpha48 or zsig97 RNA. Such tissues and cells are identified by Northern blotting (Thomas, Proc. Natl. Acad. Sci. USA 77:5201, 1980), and may include, for example, pancreas cells for zalpha48 and thyroid cells for zsig 97, although DNA can also be prepared using RNA from other tissues or isolated as genomic DNA. Total RNA can be prepared using guanidinium isothiocyanate extraction followed by isolation by centrifugation in a CsCl gradient (Chirgwin et al., Biochemistry 18:52-94, 1979). Poly (A)⁺ RNA is prepared from total RNA using the method of Aviv and Leder (Proc. Natl. Acad. Sci. USA 69:1408-12, 1972). Complementary DNA (cDNA) is prepared from poly(A)⁺ RNA using known methods. In the alternative, genomic DNA can be isolated. Polynucleotides encoding zalpha48 or zsig97 polypeptides are then identified and isolated by, for example, hybridization or PCR.

A full-length clone encoding zalpha48 or zsig97 can be obtained by conventional cloning procedures. Complementary DNA (cDNA) clones are preferred, although for some applications (e.g., expression in transgenic animals) it may be preferable to use a genomic clone, or to modify a cDNA clone to include at least one genomic intron. Methods for preparing cDNA and genomic clones are well known and within the level of ordinary skill in the art, and include the use of the sequence disclosed herein, or parts thereof, for probing or priming a library. Expression libraries can be probed with antibodies to zalpha48 or zsig97 mature peptides or to other forms of the proteins such as the preprohormone or the prohormone, receptor fragments, or other specific binding partners.

The polynucleotides of the present invention can also be synthesized using DNA synthesis machines. If chemically synthesized double stranded DNA is required for an application such as the synthesis of a DNA or a DNA fragment, then each complementary strand is made separately, for example via the phosphoramidite method known in the art. The production of short polynucleotides (60 to 80 bp) is technically straightforward and can be accomplished by synthesizing the complementary strands and then annealing them. However, for producing longer polynucleotides (longer than about 300 bp), special strategies are usually employed. For example, synthetic DNAs (double-stranded) are assembled in modular form from single-stranded fragments that are from 20 to 100 nucleotides in length. One method for building a synthetic DNA involves producing a set of overlapping, complementary oligonucleotides. Each internal section of the DNA has complementary 3′ and 5′ terminal extensions designed to base pair precisely with an adjacent section. After the DNA is assembled, the process is completed by ligating the nicks along the backbones of the two strands. In addition to the protein coding sequence, synthetic DNAs can be designed with terminal sequences that facilitate insertion into a restriction endonuclease site of a cloning vector. Alternative ways to prepare a full-length DNA are also known in the art. See Glick and Pasternak, Molecular Biotechnology, Principles & Applications of Recombinant DNA, (ASM Press, Washington, D.C. 1994); Itakura et al., Annu. Rev. Biochem. 53: 323-56, 1984 and Climie et al., Proc. Natl. Acad. Sci. USA 87:633-7, 1990.

The present invention further provides counterpart polypeptides and polynucleotides from other species (orthologs). These species include, but are not limited to mammalian, avian, amphibian, reptile, fish, insect and other vertebrate and invertebrate species. Of particular interest are zalpha48 or zsig97 polypeptides from other mammalian species, including murine, porcine, ovine, bovine, canine, feline, equine, and other primate polypeptides. The murine orthologs of zalpha48 are disclosed herein as SEQ ID NOS:3 and 4; murine orthologs of zsig97 are disclosed herein as SEQ ID NOS: 7 and 8. Additional orthologs of human zalpha48 or zsig97 can be cloned using information and compositions provided by the present invention in combination with conventional cloning techniques. For example, a cDNA can be cloned using mRNA obtained from a tissue or cell type that expresses zalpha48 or zsig97 as disclosed herein. Suitable sources of mRNA can be identified by probing Northern blots with probes designed from the sequences disclosed herein. A library is then prepared from mRNA of a positive tissue or cell line. A zalpha48- or zsig97-encoding cDNA can then be isolated by a variety of methods, such as by probing with a complete or partial human cDNA or with one or more sets of degenerate probes based on the disclosed sequences. A cDNA can also be cloned using the polymerase chain reaction, or PCR (Mullis, U.S. Pat. No. 4,683,202), using primers designed from the representative human zalpha48 or zsig97 sequence disclosed herein. Within an additional method, the cDNA library can be used to transform or transfect host cells, and expression of the cDNA of interest can be detected with an antibody to zalpha48 or zsig97 polypeptide. Similar techniques can also be applied to the isolation of genomic clones.

Those skilled in the art will recognize that the sequence disclosed in SEQ ID NOS:1 and 5 represents a single allele of human zalpha48 and zsig97, respectively, and that allelic variation and alternative splicing are expected to occur. Allelic variants of this sequence can be cloned by probing cDNA or genomic libraries from different individuals according to standard procedures. Allelic variants of the DNA sequence shown in SEQ ID NO:1 and 5, including those containing silent mutations and those in which mutations result in amino acid sequence changes, are within the scope of the present invention, as are proteins which are allelic variants of SEQ ID NO:2 and 6. cDNAs generated from alternatively spliced mRNAs, which retain the properties of the zalpha48 or zsig97 polypeptide are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art.

The present invention also provides isolated zalpha48 or zsig97 polypeptides that are substantially similar to the polypeptides of SEQ ID NO:2 or SEQ ID NO:4, respectively, and their orthologs. The term “substantially similar” is used herein to denote polypeptides having 70%, preferably 75%, more preferably at least 80%, sequence identity to the sequences shown in SEQ ID NO:2 or SEQ ID NO:4 or their orthologs. Such polypeptides will more preferably be at least 90% identical, and most preferably 95% or more identical to SEQ ID NO:2 or SEQ ID NO:4 or its orthologs.) Percent sequence identity is determined by conventional methods. See, for example, Altschul et al., Bull. Math. Bio. 48: 603-16, 1986 and Henikoff and Henikoff, Proc. Natl. Acad. Sci. USA 89:10915-9, 1992. Briefly, two amino acid sequences are aligned to optimize the alignment scores using a gap opening penalty of 10, a gap extension penalty of 1, and the “blosum 62” scoring matrix of Henikoff and Henikoff (supra.) as shown in Table 6 (amino acids are indicated by the standard one-letter codes). The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

TABLE 6

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Sequence identity of polynucleotide molecules is determined by similar methods using a ratio as disclosed above.

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative variant zalpha48 or zsig97. The FASTA algorithm is described by Pearson and Lipman, Proc. Nat'l Acad. Sci. USA 85:2444 (1988), and by Pearson, Meth. Enzymol. 183:63 (1990).

Briefly, FASTA first characterizes sequence similarity by identifying regions shared by the query sequence (e.g., SEQ ID NO:2 or SEQ ID NO:4) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Preferred parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from three to six, most preferably three, with other parameters set as default.

The BLOSUM62 table (Table 6) is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed below), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Variant zalpha48 or zsig97 polypeptides or substantially homologous zalpha48 or zsig97 polypeptides are characterized as having one or more amino acid substitutions, deletions or additions. These changes are preferably of a minor nature, that is conservative amino acid substitutions (see Table 7) and other substitutions that do not significantly affect the folding or activity of the polypeptide; small deletions, typically of one to about 30 amino acids; and amino- or carboxyl-terminal extensions, such as an amino-terminal methionine residue, a small linker peptide of up to about 20-25 residues, or an affinity tag. The present invention thus includes polypeptides of from about 14 to about 500 amino acid residues that comprise a sequence that is at least 80%, preferably at least 90%, and more preferably 95% or more identical to the corresponding region of SEQ ID NO:2. Polypeptides comprising affinity tags can further comprise a proteolytic cleavage site between the zalpha48 or zsig97 polypeptide and the affinity tag. Preferred such sites include thrombin cleavage sites and factor Xa cleavage sites.

TABLE 7
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

The present invention further provides a variety of other polypeptide fusions and related multimeric proteins comprising one or more polypeptide fusions. For example, a zalpha48 or zsig97 polypeptide can be prepared as a fusion to a dimerizing protein as disclosed in U.S. Pat. Nos. 5,155,027 and 5,567,584. Preferred dimerizing proteins in this regard include immunoglobulin constant region domains. Immunoglobulin-zalpha48 or immunoglobulin-zsig97 fusions, particularly the mature peptide sequences of these proteins, can be expressed in genetically engineered cells to produce a variety of multimeric zalpha48 or zsig97 analogs. Auxiliary domains can be fused to zalpha48 or zsig97 polypeptides (particularly the mature peptides) to target them to specific cells, tissues, or macromolecules (e.g., collagen). For example, a zalpha48 or zsig97 polypeptide or protein can be targeted to a predetermined cell type by fusing a zalpha48 or zsig97 polypeptide to a ligand that specifically binds to a receptor on the surface of the target cell. In this way, polypeptides and proteins can be targeted for therapeutic or diagnostic purposes. A zalpha48 or zsig97 polypeptide can be fused to two or more moieties, such as an affinity tag for purification and a targeting domain. Polypeptide fusions can also comprise one or more cleavage sites, particularly between domains. See, Tuan et al., Connective Tissue Research 34:1-9, 1996.

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for zalpha48 or zsig97 amino acid residues.

Essential amino acids in the polypeptides of the present invention can be identified according to procedures known in the art, such as site-directed mutagenesis or alanine-scanning mutagenesis (Cunningham and Wells, Science 244: 1081-5, 1989; Bass et al., Proc. Natl. Acad. Sci. USA 88:4498-502, 1991). In the latter technique, single alanine mutations are introduced at every residue in the molecule, and the resultant mutant molecules are tested for biological activity as disclosed below to identify amino acid residues that are critical to the activity of the molecule. See also, Hilton et al., J. Biol. Chem. 271:4699-0.708, 1996. Sites of ligand-receptor or other biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaveri et al., FEBS Lett. 309:59-64, 1992. The identities of essential amino acids can also be inferred from analysis of homologies with related polypeptide sequences or proteins.

Determination of amino acid residues that are within regions or domains that are critical to maintaining structural integrity can be determined. Within these regions one can determine specific residues that will be more or less tolerant of change and maintain the overall tertiary structure of the molecule. Methods for analyzing sequence structure include, but are not limited to, alignment of multiple sequences with high amino acid or nucleotide identity and computer analysis using available software (e.g., the Insight II® viewer and homology modeling tools; MSI, San Diego, Calif.), secondary structure propensities, binary patterns, complementary packing and buried polar interactions (Barton, Current Opin. Struct. Biol. 5:372-376, 1995 and Cordes et al., Current Opin. Struct. Biol. 6:3-10, 1996). In general, when designing modifications to molecules or identifying specific fragments determination of structure will be accompanied by evaluating activity of modified molecules.

Amino acid sequence changes are made in zalpha48 or zsig97 polypeptides so as to minimize disruption of higher order structure essential to biological activity. For example, when the zalpha48 or zsig97 polypeptides comprise one or more conserved structures, changes in amino acid residues will be made so as not to disrupt the structures and other components of the molecule where changes in conformation abate some critical function, for example, binding of the molecule to its binding partners. The effects of amino acid sequence changes can be predicted by, for example, computer modeling as disclosed herein or determined by analysis of crystal structure (see, e.g., Lapthorn et al., Nat. Struct. Biol. 2:266-268, 1995). Other techniques that are well known in the art compare folding of a variant protein to a standard molecule (e.g., the native protein). For example, comparison of the cysteine pattern in a variant and standard molecules can be made. Mass spectrometry and chemical modification using reduction and alkylation provide methods for determining cysteine residues which are associated with disulfide bonds or are free of such associations (Bean et al., Anal. Biochem. 201:216-226, 1992; Gray, Protein Sci. 2:1732-1748, 1993; and Patterson et al., Anal. Chem. 66:3727-3732, 1994). It is generally believed that if a modified molecule does not have the same disulfide bonding pattern as the standard molecule folding would be affected. Another well known and accepted method for measuring folding is circular dichroism (CD). Measuring and comparing the CD spectra generated by a modified molecule and standard molecule is routine (Johnson, Proteins 7:205-214, 1990). Crystallography is another well known method for analyzing folding and structure. Nuclear magnetic resonance (NMR), digestive peptide mapping and epitope mapping are also known methods for analyzing folding and structural similarities between proteins and polypeptides (Schaanan et al., Science 257:961-964, 1992).

The identities of essential amino acids can also be inferred from analysis of sequence similarity between family members zalpha48 and zsig97. Using methods such as “FASTA” analysis described previously, regions of high similarity are identified within a family of proteins and used to analyze amino acid sequence for conserved regions. An alternative approach to identifying a variant zalpha48 or zsig97 polynucleotide on the basis of structure is to determine whether a nucleic acid molecule encoding a potential variant zalpha48 or zsi97 polynucleotide can hybridize to a nucleic acid molecule having the nucleotide sequence of SEQ ID NO:1 or 5, respectively, as discussed above.

Other methods of identifying essential amino acids in the polypeptides of the present invention are procedures known in the art, such as site-directed mutagenesis or alanine-scanning mutagenesis (Cunningham and Wells, Science 244:1081 (1989), Bass et al., Proc. Natl. Acad. Sci. USA 88:4498 (1991), Coombs and Corey, “Site-Directed Mutagenesis and Protein Engineering,” in Proteins: Analysis and Design, Angeletti (ed.), pages 259-311 (Academic Press, Inc. 1998)). In the latter technique, single alanine mutations are introduced at every residue in the molecule, and the resultant mutant molecules are tested for biological activity as disclosed below to identify amino acid residues that are critical to the activity of the molecule. See also, Hilton et al., J. Biol. Chem. 271:4699 (1996).

The present invention also includes functional fragments of zalpha48 or zsig97 polypeptides and nucleic acid molecules encoding such functional fragments. A “functional” zalpha48 or fragment thereof defined herein is characterized by its proliferative or differentiating activity, by its ability to induce or inhibit specialized cell functions, or by its ability to bind specifically to an anti-zalpha48 antibody or zalpha48 receptor (either soluble or immobilized). A “functional” zsig97 or fragment thereof defined herein is characterized by its proliferative or differentiating activity, by its ability to induce or inhibit specialized cell functions, or by its ability to bind specifically to an anti-zsig97 antibody or zsig97 receptor (either soluble or immobilized). As previously described herein, both zalpha48 and zsig97 are characterized by several cleavage sites that generate a number of bioactive mature peptides. Thus, the present invention further provides fusion proteins encompassing: (a) polypeptide molecules comprising one or more of the of the zalpha48 or zsig97 peptides described above; and (b) functional fragments comprising one or more of these peptides. The other polypeptide portion of the fusion protein may be contributed by another peptide hormone, such as insulin, glucagon, POMC, growth hormone, neuropeptide hormones, and the like, or by a non-native and/or an unrelated secretory signal peptide that facilitates secretion of the fusion protein.

Routine deletion analyses of nucleic acid molecules can be performed to obtain functional fragments of a nucleic acid molecule that encodes a zalpha48 or zsig97 polypeptide. As an illustration, DNA molecules having the nucleotide sequence of SEQ ID NO:1 or 5 or fragments thereof, can be digested with Bal31 nuclease to obtain a series of nested deletions. These DNA fragments are then inserted into expression vectors in proper reading frame, and the expressed polypeptides are isolated and tested for zalpha48 or zsig97 activity, or for the ability to bind anti-zalpha48 or zsig97 antibodies or the zalpha48 or zsig97 receptor. One alternative to exonuclease digestion is to use oligonucleotide-directed mutagenesis to introduce deletions or stop codons to specify production of a desired zalpha48 or zsig97 fragment. Alternatively, particular fragments of a zalpha48 or zsig97 polynucleotide can be synthesized using the polymerase chain reaction.

Standard methods for identifying functional domains are well-known to those of skill in the art. For example, studies on the truncation at either or both termini of interferons have been summarized by Horisberger and Di Marco, Pharmac. Ther. 66:507 (1995). Moreover, standard techniques for functional analysis of proteins are described by, for example, Treuter et al., Molec. Gen. Genet. 240:113 (1993); Content et al., “Expression and preliminary deletion analysis of the 42 kDa 2-5A synthetase induced by human interferon,” in Biological Interferon Systems, Proceedings of ISIR-TNO Meeting on Interferon Systems, Cantell (ed.), pages 65-72 (Nijhoff 1987); Herschman, “The EGF Receptor,” in Control of Animal Cell Proliferation 1, Boynton et al., (eds.) pages 169-199 (Academic Press 1985); Coumailleau et al., J. Biol. Chem. 270:29270 (1995); Fukunaga et al., J. Biol. Chem. 270:25291 (1995); Yamaguchi et al., Biochem. Pharmacol. 50:1295 (1995); and Meisel et al., Plant Molec. Biol. 30:1 (1996).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Variants of the disclosed zalpha48 or zsig97 DNA and polypeptide sequences can be generated through DNA shuffling as disclosed by Stemmer, Nature 370:389-91, 1994, Stemmer, Proc. Natl. Acad. Sci. USA 91:10747-51, 1994 and WIPO Publication WO 97/20078. Briefly, variant DNAs are generated by in vitro homologous recombination by random fragmentation of a parent DNA followed by reassembly using PCR, resulting in randomly introduced point mutations. This technique can be modified by using a family of parent DNAs, such as allelic variants or DNAs from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed herein can be combined with high-throughput, automated screening methods to detect activity of cloned, mutagenized polypeptides in host cells. Mutagenized DNA molecules that encode active polypeptides (e.g., secreted and detected by antibodies, binding assays, or measured by a signal transduction type assay) can be recovered from the host cells and rapidly sequenced using modern equipment. These methods allow the rapid determination of the importance of individual amino acid residues in a polypeptide of interest, and can be applied to polypeptides of unknown structure.

Using the methods discussed herein, one of ordinary skill in the art can identify and/or prepare a variety of polypeptides that are substantially similar to SEQ ID NO:2 or SEQ ID NO: 6 or allelic variants thereof and retain the properties of the wild-type protein. For example, using the methods described above, one could identify a receptor binding domain on the mature peptide of zalpha48 or zsig97; an extracellular ligand-binding domain of a receptor for zalpha48 or zsig97; heterodimeric and homodimeric binding domains; other functional or structural domains; affinity tags; or other domains important for protein-protein interactions or signal transduction. Such polypeptides may also include additional polypeptide segments as generally disclosed above.

For any zalpha48 or zsig97 polypeptide, including variants and fusion proteins, one of ordinary skill in the art can readily generate a fully degenerate polynucleotide sequence encoding that variant using the information set forth in Tables 6 and 7 above.

The zalpha48 or zsig97 polypeptides of the present invention, including full-length polypeptides, N-terminal polypeptide, mature one or mature two peptides, together or individually, and the C-terminal polypeptide, described herein, biologically active fragments, and fusion polypeptides, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, John Wiley and Sons, Inc., NY, 1987.

In general, a DNA sequence encoding a zalpha48 or zsig97 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a zalpha48 or zsig97 polypeptide into the secretory pathway of a host cell, a secretory signal sequence (also known as a leader sequence, prepro sequence or pre sequence) is provided in the expression vector. The secretory signal sequence may be that of zalpha48 or zsig97, or may be derived from another secreted protein (e.g., t-PA) or synthesized de novo. The secretory signal sequence is operably linked to the zalpha48 or zsig97 DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain secretory signal sequences may be positioned elsewhere in the DNA sequence of interest (see, e.g., Welch et al., U.S. Pat. No. 5,037,743; Holland et al., U.S. Pat. No. 5,143,830).

Alternatively, the secretory signal sequence contained in the polypeptides of the present invention is used to direct other polypeptides into the secretory pathway. The present invention provides for such fusion polypeptides. A signal fusion polypeptide can be made wherein a secretory signal sequence derived from zalpha48 or zsig97 is operably linked to a DNA sequence encoding another polypeptide using methods known in the art and disclosed herein. The secretory signal sequence contained in the fusion polypeptides of the present invention is preferably fused amino-terminally to an additional peptide to direct the additional peptide into the secretory pathway. Such constructs have numerous applications known in the art. For example, these novel secretory signal sequence fusion constructs can direct the secretion of an active component of a normally non-secreted protein. Such fusions may be used in vivo or in vitro to direct peptides through the secretory pathway.

Cultured mammalian cells are suitable hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-5, 1982), DEAE-dextran mediated transfection (Ausubel et al., ibid.), and liposome-mediated transfection (Hawley-Nelson et al., Focus 15:73, 1993; Ciccarone et al., Focus 15:80, 1993, and viral vectors (Miller and Rosman, BioTechniques 7:980-90, 1989; Wang and Finer, Nature Med. 2:714-6, 1996). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection, Manassas, Va. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g. hygromycin resistance, multi-drug resistance, puromycin acetyltransferase) can also be used. Alternative markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

Other higher eukaryotic cells can also be used as hosts, including plant cells, insect cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463. Insect cells can be infected with recombinant baculovirus, commonly derived from Autographa californica nuclear polyhedrosis virus (AcNPV). See, King, L. A. and Possee, R. D., The Baculovirus Expression System: A Laboratory Guide, London, Chapman & Hall; O'Reilly, D. R. et al., Baculovirus Expression Vectors: A Laboratory Manual, New York, Oxford University Press., 1994; and, Richardson, C. D., Ed., Baculovirus Expression Protocols. Methods in Molecular Biology, Totowa, N.J., Humana Press, 1995. The second method of making recombinant baculovirus utilizes a transposon-based system described by Luckow (Luckow, V. A, et al., J Virol 67:4566-79, 1993). This system is sold in the Bac-to-Bac™ kit (Life Technologies, Rockville, Md.). This system utilizes a transfer vector, pFastBac1™ (Life Technologies) containing a Tn7 transposon to move the DNA encoding the zalpha48 or zsig97 polypeptide into a baculovirus genome maintained in E. coli as a large plasmid called a “bacmid.” The pFastBac1™ transfer vector utilizes the AcNPV polyhedrin promoter to drive the expression of the gene of interest, in this case zalpha48 or zsig97. However, pFastBac™ can be modified to a considerable degree. The polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins. See, Hill-Perkins, M. S. and Possee, R. D., J. Gen. Virol. 71:971-6, 1990; Bonning, B. C. et al., J. Gen. Virol. 75:1551-6, 1994; and, Chazenbalk, G. D., and Rapoport, B., J. Biol. Chem. 270:1543-9, 1995. In such transfer vector constructs, a short or long version of the basic protein promoter can be used. Moreover, transfer vectors can be constructed which replace the native zalpha48 or zsig97 secretory signal sequences with secretory signal sequences derived from insect proteins. For example, a secretory signal sequence from Ecdysteroid Glucosyltransferase (EGT), honey bee Melittin (Invitrogen, Carlsbad, Calif.), or baculovirus gp67 (PharMingen, San Diego, Calif.) can be used in constructs to replace the native zalpha48 or zsig97 secretory signal sequence. In addition, transfer vectors can include an in-frame fusion with DNA encoding an epitope tag at the C- or N-terminus of the expressed zalpha48 or zsig97 polypeptide, for example, a Glu-Glu epitope tag (Grussenmeyer, T. et al., Proc. Natl. Acad. Sci. 82:7952-4, 1985). Using a technique known in the art, a transfer vector containing zalpha48 or zsig97 is transformed into E. Coli, and screened for bacmids which contain an interrupted lacZ gene indicative of recombinant baculovirus. The bacmid DNA containing the recombinant baculovirus genome is isolated, using common techniques, and used to transfect Spodoptera frugiperda cells, e.g. Sf9 cells. Recombinant virus that expresses zalpha48 or zsig97 peptides is subsequently produced. Recombinant viral stocks are made by methods commonly used the art.

The recombinant virus is used to infect host cells, typically a cell line derived from the fall armyworm, Spodoptera frugiperda. See, in general, Glick and Pasternak, Molecular Biotechnology: Principles and Applications of Recombinant DNA, ASM Press, Washington, D.C., 1994. Another suitable cell line is the High FiveO™ cell line (Invitrogen) derived from Trichoplusia ni (U.S. Pat. No. 5,300,435). Commercially available serum-free media are used to grow and maintain the cells. Suitable media are Sf900 II™ (Life Technologies) or ESF 921™ (Expression Systems) for the Sf9 cells; and Ex-cellO405™ (JRH Biosciences, Lenexa, Kans.) or Express FiveO™ (Life Technologies) for the T. ni cells. The cells are grown up from an inoculation density of approximately 2-5×10⁵ cells to a density of 1-2×10⁶ cells at which time a recombinant viral stock is added at a multiplicity of infection (MOI) of 0.1 to 10, more typically near 3. Procedures used are generally described in available laboratory manuals (King, L. A. and Possee, R. D., ibid.; O'Reilly, D. R. et al., ibid.; Richardson, C. D., ibid.). Subsequent purification of the zalpha48 or zsig97 polypeptide from the supernatant can be achieved using methods described herein.

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-65, 1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

The use of Pichia methanolica as host for the production of recombinant proteins is disclosed in WIPO Publications WO 97/17450, WO 97/17451, WO 98/02536, and WO 98/02565. DNA molecules for use in transforming P. methanolica will commonly be prepared as double-stranded, circular plasmids, which are preferably linearized prior to transformation. For polypeptide production in P. methanolica, it is preferred that the promoter and terminator in the plasmid be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes. To facilitate integration of the DNA into the host chromosome, it is preferred to have the entire expression segment of the plasmid flanked at both ends by host DNA sequences. A preferred selectable marker for use in Pichia methanolica is a P. methanolica ADE2 gene, which encodes phosphoribosyl-5-aminoimidazole carboxylase (AIRC; EC 4.1.1.21), which allows ade2 host cells to grow in the absence of adenine. For large-scale, industrial processes where it is desirable to minimize the use of methanol, it is preferred to use host cells in which both methanol utilization genes (AUG1 and AUG2) are deleted. For production of secreted proteins, host cells deficient in vacuolar protease genes (PEP4 and PRB1) are preferred. Electroporation is used to facilitate the introduction of a plasmid containing DNA encoding a polypeptide of interest into P. methanolica cells. It is preferred to transform P. methanolica cells by electroporation using an exponentially decaying, pulsed electric field having a field strength of from 2.5 to 4.5 kV/cm, preferably about 3.75 kV/cm, and a time constant (t) of from 1 to 40 milliseconds, most preferably about 20 milliseconds.

Prokaryotic host cells, including strains of the bacteria Escherichia coli, Bacillus and other genera are also useful host cells within the present invention.

Techniques for transforming these hosts and expressing foreign DNA sequences cloned therein are well known in the art (see, e.g., Sambrook et al., ibid.). When expressing a zalpha48 or zsig97 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell. P. methanolica cells are cultured in a medium comprising adequate sources of carbon, nitrogen and trace nutrients at a temperature of about 25° C. to 35° C. Liquid cultures are provided with sufficient aeration by conventional means, such as shaking of small flasks or sparging of fermentors. A preferred culture medium for P. methanolica is YEPD (2% D-glucose, 2% Bacto™ Peptone (Difco Laboratories, Detroit, Mich.), 1% Bacto™ yeast extract (Difco Laboratories), 0.004% adenine and 0.006% L-leucine).

It is preferred to purify the polypeptides of the present invention to ≧80% purity, more preferably to ≧90% purity, even more preferably ≧95% purity, and particularly preferred is a pharmaceutically pure state, that is greater than 99.9% pure with respect to contaminating macromolecules, particularly other proteins and nucleic acids, and free of infectious and pyrogenic agents. Preferably, a purified polypeptide is substantially free of other polypeptides, particularly other polypeptides of animal origin.

Expressed recombinant zalpha48 or zsig97 polypeptides (or chimeric zalpha48 or zsig97 polypeptides) can be purified using fractionation and/or conventional purification methods and media. Ammonium sulfate precipitation and acid or chaotrope extraction may be used for fractionation of samples. Exemplary purification steps can include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties. Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Methods for binding receptor polypeptides to support media are well known in the art. Selection of a particular method is a matter of routine design and is determined in part by the properties of the chosen support. See, for example, Affinity Chromatography: Principles & Methods, Pharmacia LKB Biotechnology, Uppsala, Sweden, 1988.

The polypeptides of the present invention can be isolated by exploitation of their structural and biological properties. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., Vol. 182, “Guide to Protein Purification”, M. Deutscher, (ed.), Acad. Press, San Diego, 1990, pp. 529-39). Within additional embodiments of the invention, a fusion of the polypeptide of interest and an affinity tag (e.g., maltose-binding protein, an immunoglobulin domain) may be constructed to facilitate purification.

Moreover, using methods described in the art, polypeptide fusions, or hybrid zalpha48 or zsig97 proteins, are constructed using regions or domains of zalpha48 or zsig97 in combination with those of paralogs, orthologs, or heterologous proteins (Sambrook et al., ibid., Altschul et al., ibid., Picard. D., Cur. Opin. Biology, 5:511-515, 1994, and references therein). These methods allow the determination of the biological importance of larger domains or regions in a polypeptide of interest. Such hybrids may alter reaction kinetics, binding, constrict or expand the substrate specificity, or alter tissue and cellular localization of a polypeptide, and can be applied to polypeptides of unknown structure.

Fusion polypeptides can be prepared by methods known to those skilled in the art by preparing each component of the fusion protein and chemically conjugating them. Alternatively, a polynucleotide encoding one or more components of the fusion protein in the proper reading frame can be generated using known techniques and expressed by the methods described herein. For example, part or all of a domain(s) conferring a biological function may be swapped between zalpha48 or zsig97 of the present invention with the functionally equivalent domain(s) from another family member. Such domains include, but are not limited to the secretory signal sequence, mature one and mature two peptides, described herein. Such fusion proteins would be expected to have a biological functional profile that is the same or similar to polypeptides of the present invention or other known family proteins or to a heterologous protein, depending on the fusion constructed. Moreover, such fusion proteins may exhibit other properties as disclosed herein.

Standard molecular biological and cloning techniques can be used to swap the equivalent domains between the zalpha48 or zsig97 polypeptide and those polypeptides to which they are fused. Generally, a DNA segment that encodes a domain of interest, e.g., a zalpha48 or zsig97 mature one or mature two peptide, is operably linked in frame to at least one other DNA segment encoding an additional polypeptide and inserted into an appropriate expression vector, as described herein. Generally DNA constructs are made such that the several DNA segments that encode the corresponding regions of a polypeptide are operably linked in frame to make a single construct that encodes the entire fusion protein, or a functional portion thereof. For example, a DNA construct would encode from N-terminus to C-terminus a fusion protein comprising a signal polypeptide followed by a mature polypeptide; or a DNA construct would encode from N-terminus to C-terminus a fusion protein, with or without a signal polypeptide, comprising an N-terminal polypeptide, followed by mature one and mature two pepetide, and the C-terminal polypeptide, or as interchanged with equivalent regions from another protein. Such fusion proteins can be expressed, isolated, and assayed for activity as described herein. Moreover, such fusion proteins can be used to express and secrete fragments of the zalpha48 or zsig97 polypeptide, to be used, for example to inoculate an animal to generate anti-zalpha48 or zsig97 antibodies as described herein. For example a secretory signal sequence can be operably linked to the mature one or mature peptide or a combination thereof to secrete a fragment of zalpha48 or zsig97 polypeptide that can be purified as described herein and serve as an antigen to be inoculated into an animal to produce anti-zalpha48 or zsig97 antibodies, as described herein.

Protein refolding (and optionally reoxidation) procedures may be advantageously used. Zalpha48 or zsig97 polypeptides or fragments thereof may also be prepared through chemical synthesis. Zalpha48 or zsig97 polypeptides may be monomers or multimers; glycosylated or non-glycosylated; pegylated or non-pegylated; and the preprohormone may or may not include an initial methionine amino acid residue.

Polypeptides of the present invention can also be synthesized by exclusive solid phase synthesis, partial solid phase methods, fragment condensation or classical solution synthesis. Methods for synthesizing polypeptides are well known in the art. See, for example, Merrifield, J. Am. Chem. Soc. 85:2149, 1963; Kaiser et al., Anal. Biochem. 34:595, 1970. After the entire synthesis of the desired peptide on a solid support, the peptide-resin is washed with a reagent which cleaves the polypeptide from the resin and removes most of the side-chain protecting groups. Such methods are well established in the art.

The activity of molecules of the present invention can be measured using a variety of assays that measure cell differentiation and proliferation as well as assays that measure cell contractility and cardiovascular function. Such assays are well known in the art.

Several tissues in which zalpha48 is highly and moderately expressed are glandular and reproductive tissues. For example, tissues in which zalpha48 is expressed include cells of the pancreas, adrenal gland, ovary, and pituitary. Like other peptide hormones, the mature peptides may also enter the bloodsteam and act upon tissues far removed from the location of its expression such as neural tissue. The effects of zalpha48 polypeptides, its antagonists and agonists, can be measured in vitro using cellular assay systems. Such assays are known in the art and can be applied to tissue samples as well as to organ systems and can be used to determine whether zalpha48 polypeptide, its agonists or antagonists, have an effect on glandular function, reproductive function, or neural function. Molecules of the present invention are hence useful for treating dysfunction associated with glandular, reproductive, or neural tissues. As such, molecules of the present invention have utility in treating digestive problems, growth dysfunction, nervous disease, infertility, and other hormonal diseases, and could aid in in vitro fertilization and birth control.

Several tissues in which zsig97 is highly and moderately expressed are glandular, neural, and immunological tissues. For example, tissues in which zsig97 is expressed include cells in the thyroid, prostate (both gland and smooth muscle cells), dendritic cells, and lymphcytes such as monocytes. Like other peptide hormones, the mature peptides may also enter the bloodsteam and act upon tissues far removed from the location of its expression. The effects of zsig97 polypeptides, its antagonists and agonists, can be measured in vitro using cellular assay systems. Such assays are known in the art and can be applied to tissue samples as well as to organ systems and can be used to determine whether zsig97 polypeptide, its agonists or antagonists, have an effect on glandular, neural, or immunologic function. Molecules of the present invention are hence useful for treating dysfunction associated with glandular, neural, or immunologic tissues. As such, molecules of the present invention have utility in treating glandular dysfunction, nervous disease, and immunological problems.

Many peptide hormones, such as those within family of gut-brain peptides, are associated with neurological and CNS functions as well as cardiovascular functions. For example, NPY, a peptide with receptors in both the brain and the gut has been shown to stimulate appetite when administered to the central nervous system (Gehlert, Life Sciences 55(6):551-562, 1994). Moreover, NPY has been implicated in cardiovascular effects such as increased sympathetic nerve activity in heart, which is associated with heart failure, as well as hypotension, and changes in blood pressure and vagal action (Feng, Q. et al Acta. Physiol. Scand. 166:285-291, 1999; McLean, K J. Et al. Neuroscience 92:1377-1387, 1999; Potter, E K et al; Regul. Pept. 25:167-177, 1989; Gardiner, S M Brain Res. Brain Res. Review 14:79-116, 1989). Moreover, other peptide hormones such as motilin, have immunoreactivity identified in different regions of the brain, particularly the cerebellum, and in the pituitary (Gasparini et al., Hum. Genetics 94(6):671-674, 1994). Motilin has been found to coexist with neurotransmitter γ-aminobutyric acid in cerebellum (Chan-Patay, Proc. Sym. 50th Anniv. Meet. Br. Pharmalog. Soc.:1-24, 1982). Physiological studies have provided some evidence that motilin has an affect on feeding behavior (Rosenfield et al., Phys. Behav. 39(6):735-736, 1987), bladder control, pituitary growth hormone release.

Examples such as NPY and motilin emphasize the importance and broad activity of peptide hormones in the human body, and their impact on normal physiological function and disease. Peptide hormones are involved in regulatory aspects of cardiovascular regulation and homeostasis, digestion, brain, neuronal and other organ functions. Various peptide hormones have been shown to be involved in control of blood pressure, heart rate, arrhythmia, osmotic balance, influencing the release and action of cardiovascular transmitters, vasoconstriction and vasodilatation, vasoconstriction resulting in myocardial ischemia, vasomotor tone, contractility, food intake, respiration, behavior, and pain modulation, and the like. As a peptide hormone, zalpha48 or zsig97 may similarly exert effects in thyroid, pancreas, or other tissues in which it is expressed, or freely circulate through the body and exert effects elsewhere. Thus, zalpha48 or zsig97 peptides can regulate positively or negatively various physiological functions, or cause the release of other regulatory hormones from glands, such as the thyroid, prostate, or pancreas, CNS and other organs or tissues. Assays and models to test for such zalpha48 or zsig97 activity are well known in the art and described herein.

Moreover, immunohistochemical and immunolabeling methods known in the art and described herein can be used to assess zalpha48 or zsig97 polypeptide and peptide influence on the release and of glandular effectors and other function, as well as interactions between zalpha48 or zsig97 polypeptides and peptides with other peptide effectors, such as VIP, NPY and other peptides (Wharton, J, and Gulbenkian S. Experientia Suppl. 56:292-316, 1989; and Forsgren, S. Cell Tissue Res. 256:125-135, 1989). As such, labeled inventive zalpha48 or zsig97 polypeptides, peptides, and antibodies can be used to assess these interactions. In addition, such labeled zalpha48 or zsig97 polypeptides, peptides, and antibodies can be used as diagnostics to assess human disease in comparison to normal controls, and described herein. Such histologic, immunohistochemical and immunolabeling methods and the like can be used in conjunction with the in vivo models described above and herein.

Proteins of the present invention are useful for example, in treating reproductive, prostate, pituitary, pancreatic, thyroid, neural, ovary, and other disorders, and can be measured in vitro using cultured cells or in vivo by administering molecules of the present invention to the appropriate animal model. For instance, host cells expressing a zalpha48 or zsig97 polypeptide can be embedded in an alginate environment and injected (implanted) into recipient animals. Alginate-poly-L-lysine microencapsulation, permselective membrane encapsulation and diffusion chambers are a means to entrap transfected mammalian cells or primary mammalian cells. These types of non-immunogenic “encapsulations” permit the diffusion of proteins and other macromolecules secreted or released by the captured cells to the recipient animal. Most importantly, the capsules mask and shield the foreign, embedded cells from the recipient animal's immune response. Such encapsulations can extend the life of the injected cells from a few hours or days (naked cells) to several weeks (embedded cells). Alginate threads provide a simple and quick means for generating embedded cells.

The materials needed to generate the alginate threads are known in the art. In an exemplary procedure, 3% alginate is prepared in sterile H₂O, and sterile filtered. Just prior to preparation of alginate threads, the alginate solution is again filtered. An approximately 50% cell suspension (containing about 5×10⁵ to about 5×10⁷ cells/ml) is mixed with the 3% alginate solution. One ml of the alginate/cell suspension is extruded into a 100 mM sterile filtered CaCl₂ solution over a time period of ˜15 min, forming a “thread”. The extruded thread is then transferred into a solution of 50 mM CaCl₂, and then into a solution of 25 mM CaCl₂. The thread is then rinsed with deionized water before coating the thread by incubating in a 0.01% solution of poly-L-lysine. Finally, the thread is rinsed with Lactated Ringer's Solution and drawn from solution into a syringe barrel (without needle). A large bore needle is then attached to the syringe, and the thread is intraperitoneally injected into a recipient in a minimal volume of the Lactated Ringer's Solution.

An in vivo approach for assaying proteins of the present invention involves viral delivery systems. Exemplary viruses for this purpose include adenovirus, herpesvirus, retroviruses, vaccinia virus, and adeno-associated virus (AAV). Adenovirus, a double-stranded DNA virus, is currently the best studied gene transfer vector for delivery of heterologous nucleic acid (for review, see T. C. Becker et al., Meth. Cell Biol. 43:161-89, 1994; and J. T. Douglas and D. T. Curiel, Science & Medicine 4:44-53, 1997). The adenovirus system offers several advantages: (i) adenovirus can accommodate relatively large DNA inserts; (ii) can be grown to high-titer; (iii) infect a broad range of mammalian cell types; and (iv) can be used with many different promoters including ubiquitous, tissue specific, and regulatable promoters. Also, because adenoviruses are stable in the bloodstream, they can be administered by intravenous injection.

Using adenovirus vectors where portions of the adenovirus genome are deleted, inserts are incorporated into the viral DNA by direct ligation or by homologous recombination with a co-transfected plasmid. In an exemplary system, the essential E1 gene has been deleted from the viral vector, and the virus will not replicate unless the E1 gene is provided by the host cell (the human 293 cell line is exemplary). When intravenously administered to intact animals, adenovirus primarily targets the liver. If the adenoviral delivery system has an E1 gene deletion, the virus cannot replicate in the host cells. However, the host's tissue (e.g., liver) will express and process (and, if a secretory signal sequence is present, secrete) the heterologous protein. Secreted proteins will enter the circulation in the highly vascularized liver, and effects on the infected animal can be determined.

Moreover, adenoviral vectors containing various deletions of viral genes can be used in an attempt to reduce or eliminate immune responses to the vector. Such adenoviruses are E1 deleted, and in addition contain deletions of E2A or E4 (Lusky, M. et al., J. Virol. 72:2022-2032, 1998; Raper, S. E. et al., Human Gene Therapy 9:671-679, 1998). In addition, deletion of E2b is reported to reduce immune responses (Amalfitano, A. et al., J. Virol. 72:926-933, 1998). Moreover, by deleting the entire adenovirus genome, very large inserts of heterologous DNA can be accommodated. Generation of so called “gutless” adenoviruses where all viral genes are deleted are particularly advantageous for insertion of large inserts of heterologous DNA. For review, see Yeh, P. and Perricaudet, M., FASEB J. 11:615-623, 1997.

The adenovirus system can also be used for protein production in vitro. By culturing adenovirus-infected non-293 cells under conditions where the cells are not rapidly dividing, the cells can produce proteins for extended periods of time. For instance, BHK cells are grown to confluence in cell factories, then exposed to the adenoviral vector encoding the secreted protein of interest. The cells are then grown under serum-free conditions, which allows infected cells to survive for several weeks without significant cell division. Alternatively, adenovirus vector infected 293 cells can be grown as adherent cells or in suspension culture at relatively high cell density to produce significant amounts of protein (See Garnier et al., Cytotechnol. 15:145-55, 1994). With either protocol, an expressed, secreted heterologous protein can be repeatedly isolated from the cell culture supernatant, lysate, or membrane fractions depending on the disposition of the expressed protein in the cell. Within the infected 293 cell production protocol, non-secreted proteins may also be effectively obtained.

As a ligand, the activity of zalpha48 or zsig97 polypeptide can be measured by a silicon-based biosensor microphysiometer which measures the extracellular acidification rate or proton excretion associated with receptor binding and subsequent physiologic cellular responses. An exemplary device is the Cytosensor™ Microphysiometer manufactured by Molecular Devices, Sunnyvale, Calif. A variety of cellular responses, such as cell proliferation, ion transport, energy production, inflammatory response, regulatory and receptor activation, and the like, can be measured by this method. See, for example, McConnell, H. M. et al., Science 257:1906-1912, 1992; Pitchford, S. et al., Meth. Enzymol. 228:84-108, 1997; Arimilli, S. et al., J. Immunol. Meth. 212:49-59, 1998; Van Liefde, I. et al., Eur. J. Pharmacol. 346:87-95, 1998. The microphysiometer can be used for assaying adherent or non-adherent eukaryotic or prokaryotic cells. By measuring extracellular acidification changes in cell media over time, the microphysiometer directly measures cellular responses to various stimuli, including zalpha48 or zsig97 polypeptide, its agonists, or antagonists. Preferably, the microphysiometer is used to measure responses of a zalpha48- or zsig97-responsive eukaryotic cell, compared to a control eukaryotic cell that does not respond. Zalpha48 or zsig97-responsive eukaryotic cells comprise cells into which a receptor for zalpha48 or zsig97 has been transfected creating a cell that is responsive to zalpha48 or zsig97 mature peptide one or two; or cells naturally responsive to zalpha48 or zsig97 such as cells derived from prostate, thyroid, pancrease, ovary, pituitary, or the like. Differences, measured by a change, for example, an increase or diminution in extracellular acidification, in the response of cells exposed to zalpha48 or zsig97 mature peptide one or two, relative to a control not exposed to zalpha48 or zsig97 mature peptide, are a direct measurement of zalpha48 or zsig97-modulated cellular responses. Moreover, such zalpha48 or zsig97-modulated responses can be assayed under a variety of stimuli. Using the microphysiometer, there is provided a method of identifying agonists of zalpha48 or zsig97 polypeptide, comprising providing cells responsive to a zalpha48 or zsig97 polypeptide, culturing a first portion of the cells in the absence of a test compound, culturing a second portion of the cells in the presence of a test compound, and detecting a change, for example, an increase or diminution, in a cellular response of the second portion of the cells as compared to the first portion of the cells. The change in cellular response is shown as a measurable change extracellular acidification rate. Moreover, culturing a third portion of the cells in the presence of zalpha48 or zsig97 polypeptide and the absence of a test compound can be used as a positive control for the zalpha48 or zsig97-responsive cells, and as a control to compare the agonist activity of a test compound with that of the zalpha48 or zsig97 polypeptide. Moreover, using the microphysiometer, there is provided a method of identifying antagonists of zalpha48 or zsig97 polypeptide, comprising providing cells responsive to a zalpha48 or zsig97 polypeptide, culturing a first portion of the cells in the presence of zalpha48 or zsig97 and the absence of a test compound, culturing a second portion of the cells in the presence of zalpha48 or zsig97 and the presence of a test compound, and detecting a change, for example, an increase or a diminution in a cellular response of the second portion of the cells as compared to the first portion of the cells. The change in cellular response is shown as a measurable change extracellular acidification rate. Antagonists and agonists, for zalpha48 or zsig97 polypeptide, can be rapidly identified using this method.

Moreover, zalpha48 or zsig97 can be used to identify cells, tissues, or cell lines which respond to a zalpha48 or zsig97-stimulated pathway. The microphysiometer, described above, can be used to rapidly identify ligand-responsive cells, such as cells responsive to zalpha48 or zsig97 of the present invention. Cells can be cultured in the presence or absence of zalpha48 or zsig97 polypeptide. Those cells which elicit a measurable change in extracellular acidification in the presence of zalpha48 or zsig97 are responsive to zalpha48 or zsig97. Such cell lines, can be used to identify antagonists and agonists of zalpha48 or zsig97 polypeptide as described above.

In view of the tissue distribution observed for zalpha48 or zsig97 polypeptides, agonists (including the natural ligand/substrate/cofactor/etc.) and antagonists have enormous potential in both in vitro and in vivo applications. For example, zalpha48 or zsig97 polypeptide and agonist compounds are useful as components of defined cell culture media, and may be used alone or in combination with cytokines and hormones to replace serum that is commonly used in cell culture. Agonists are thus useful in specifically promoting the growth and/or development of mammalian cells in vitro, particularly of those derived from reproductive tissues. As such, zalpha48 or zsig97 polypeptides or agonists are added to tissue culture media for these cell types.

Zalpha48 or zsig97 can also be used to identify inhibitors (antagonists) of its activity. Test compounds are added to assays disclosed herein to identify compounds that inhibit the activity of zalpha48 or zsig97. In addition to those assays disclosed herein, samples can be tested for inhibition of zalpha48 or zsig97 activity within a variety of assays designed to measure receptor binding or the stimulation/inhibition of zalpha48 or zsig97-dependent cellular responses. For example, zalpha48 or zsig97-responsive cell lines can be transfected with a reporter gene construct that is responsive to a zalpha48 or zsig97-stimulated cellular pathway. Reporter gene constructs of this type are known in the art, and will generally comprise a zalpha48 or zsig97-DNA response element operably linked to a gene encoding an assayable protein, such as luciferase. DNA response elements can include, but are not limited to, cyclic AMP response elements (CRE), hormone response elements (HRE) insulin response element (IRE) (Nasrin et al., Proc. Natl. Acad. Sci. USA 87:5273-7, 1990) and serum response elements (SRE) (Shaw et al. Cell 56: 563-72, 1989). Cyclic AMP response elements are reviewed in Roestler et al., J. Biol. Chem. 263 (19):9063-6; 1988 and Habener, Molec. Endocrinol. 4 (8): 1087-94; 1990. Hormone response elements are reviewed in Beato, Cell 56:335-44; 1989. Candidate compounds, solutions, mixtures or extracts are tested for the ability to inhibit the activity of zalpha48 or zsig97 on the target cells as evidenced by a decrease in zalpha48 or zsig97 stimulation of reporter gene expression. Assays of this type will detect compounds that directly block zalpha48 or zsig97 binding to cell-surface receptors, as well as compounds that block processes in the cellular pathway subsequent to receptor-ligand binding. In the alternative, compounds or other samples can be tested for direct blocking of zalpha48 or zsig97 binding to receptor using zalpha48 or zsig97 tagged with a detectable label (e.g., ¹²⁵I, biotin, horseradish peroxidase, FITC, and the like). Within assays of this type, the ability of a test sample to inhibit the binding of labeled zalpha48 or zsig97 to the receptor is indicative of inhibitory activity, which can be confirmed through secondary assays. Receptors used within binding assays may be cellular receptors or isolated, immobilized receptors.

As a secreted peptide hormone, zalpha48 or zsig97 may play a role in reproduction, as it is produced in various reproductive organs such as the prostate, the ovary, and the pituitary. In view of the tissue specificity observed for zalpha48 or zsig97, agonists and antagonists have enormous potential in both in vitro and in vivo applications. Zalpha48 or zsig97 polypeptides, agonists and antagonists may also prove useful in modulating reproductive cycles and thus aid in overcoming infertility.

Antagonists are useful as research reagents for characterizing sites of ligand-receptor interaction. In vivo, zalpha48 or zsig97 polypeptides, agonists or antagonists may find application in the diagnosis or treatment of female infertility or as a female contraceptive agents.

Accordingly, proteins of the present invention can have applications in enhancing fertilization during assisted reproduction in humans and in animals. Such assisted reproduction methods are known in the art and include artificial insemination, in vitro fertilization, embryo transfer and gamete intrafallopian transfer. Such methods are useful for assisting men and women who have physiological or metabolic disorders preventing natural conception or can be used to enhance in vitro fertilization. Such methods are also used in animal breeding programs, such as for livestock breeding and could be used as methods for the creation of transgenic animals. Proteins of the present invention can be combined with sperm, an egg or an egg-sperm mixture prior to fertilization of the egg. In some species, sperm capacitate spontaneously during in vitro fertilization procedures, but normally sperm capacitate over an extended period of time both in vivo and in vitro. It is advantageous to increase sperm activation during such procedures to enhance the likelihood of successful fertilization. The washed sperm or sperm removed from the seminal plasma used in such assisted reproduction methods has been shown to have altered reproductive functions, in particular, reduced motility and zona interaction. To enhance fertilization during assisted reproduction methods sperm is capacitated using exogenously added compounds.

In cases where pregnancy is not desired, zalpha48 or zsig97 polypeptide or polypeptide fragments may function as germ-cell-specific antigens for use as components in “immunocontraceptive” or “anti-fertility” vaccines to induce formation of antibodies and/or cell mediated immunity to selectively inhibit a process, or processes, critical to successful reproduction in humans and animals.

Regulation of reproductive function in males and females is controlled in part by feedback inhibition of the hypothalamus and anterior pituitary by blood-borne hormones. Testis proteins, such as activins and inhibins, have been shown to regulate secretion of active molecules including follicle stimulating hormone (FSH) from the pituitary (Ying, Endodcr. Rev. 9:267-93, 1988; Plant et al., Hum. Reprod. 8:41-44,1993). Inhibins, also expressed in the ovaries, have been shown to regulate ovarian functions (Woodruff et al., Endocr. 132:2332-42,1993; Russell et al., J. Reprod. Fertil. 100:115-22, 1994). Relaxin has been shown to be a systemic and local acting hormone regulating follicular and uterine growth (Bagnell et al., J. Reprod. Fertil. 48:127-38, 1993). As such, the polypeptides of the present invention may also have effects on female gametes and reproductive tract. These functions may also be associated with zalpha48 or zsig97 polypeptides and may be used to regulate ovarian functions.

A zalpha48 or zsig97 polypeptide can be expressed as a fusion with an immunoglobulin heavy chain constant region, typically an Fc fragment, which contains two constant region domains and lacks the variable region. Methods for preparing such fusions are disclosed in U.S. Pat. Nos. 5,155,027 and 5,567,584. Such fusions are typically secreted as multimeric molecules wherein the Fc portions are disulfide bonded to each other and two non-Ig polypeptides are arrayed in closed proximity to each other. Fusions of this type can be used as drug-delivery devices, to stimulate a zalpha48 or zsig97-induced signal transduction cascade in vivo or in vitro, or to affinity purify zalpha48 or zsig97 receptors, as in vitro assay tool, or as an antagonist. For use in assays, the chimeras are bound to a support via the Fc region and used in an ELISA format.

A zalpha48 or zsig97 ligand-binding polypeptide can also be used for purification of ligand. The polypeptide is immobilized on a solid support, such as agarose beads, cross-linked agarose, glass, cellulosic resins, silica-based resins, polystyrene, cross-linked polyacrylamide, or like materials that are stable under the conditions of use. Methods for linking polypeptides to solid supports are known in the art, and include amine chemistry, cyanogen bromide activation, N-hydroxysuccinimide activation, epoxide activation, sulfhydryl activation, and hydrazide activation. The resulting medium will generally be configured in the form of a column, and fluids containing ligand are passed through the column one or more times to allow ligand to bind to the receptor polypeptide. The ligand is then eluted using changes in salt concentration, chaotropic agents (guanidine HCl), or pH to disrupt ligand-receptor binding.

An assay system that uses a ligand-binding receptor (or an antibody, one member of a complement/anti-complement pair) or a binding fragment thereof, and a commercially available biosensor instrument (BIAcore, Pharmacia Biosensor, Piscataway, N.J.) may be advantageously employed. Such receptor, antibody, member of a complement/anti-complement pair or fragment is immobilized onto the surface of a receptor chip. Use of this instrument is disclosed by Karlsson, J. Immunol. Methods 145:229-40, 1991 and Cunningham and Wells, J. Mol. Biol. 234:554-63, 1993. A receptor, antibody, member or fragment is covalently attached, using amine or sulfhydryl chemistry, to dextran fibers that are attached to gold film within the flow cell. A test sample is passed through the cell. If a ligand, epitope, or opposite member of the complement/anti-complement pair is present in the sample, it will bind to the immobilized receptor, antibody or member, respectively, causing a change in the refractive index of the medium, which is detected as a change in surface plasmon resonance of the gold film. This system allows the determination of on- and off-rates, from which binding affinity can be calculated, and assessment of stoichiometry of binding.

Ligand-binding receptor polypeptides can also be used within other assay systems known in the art. Such systems include Scatchard analysis for determination of binding affinity (see Scatchard, Ann. NY Acad. Sci. 51: 660-72, 1949) and calorimetric assays (Cunningham et al., Science 253:545-48, 1991; Cunningham et al., Science 245:821-25, 1991).

Zalpha48 or zsig97 polypeptides can also be used to prepare antibodies that bind to zalpha48 or zsig97 epitopes, peptides or polypeptides. The zalpha48 or zsig97 polypeptide or a fragment thereof serves as an antigen (immunogen) to inoculate an animal and elicit an immune response. One of skill in the art would recognize that antigenic, epitope-bearing polypeptides contain a sequence of at least 6, preferably at least 9, and more preferably at least 15 to about 30 contiguous amino acid residues of a zalpha48 or zsig97 polypeptide (e.g., SEQ ID NO:2 or 6). Polypeptides comprising a larger portion of a zalpha48 or zsig97 polypeptide, i.e., from 10 to 30 residues up to the entire length of the amino acid sequence are included. Antigens or immunogenic epitopes can also include attached tags, adjuvants and carriers, as described herein. Suitable antigens include the zalpha48 or zsig97 polypeptide encoded by SEQ ID NO:2 from amino acid number 1 (Met) to amino acid number 151 (His), or a contiguous 20 to 151 amino acid fragment thereof. Other suitable antigens include the mature one or mature two peptide disclosed herein. Antibodies from an immune response generated by inoculation of an animal with these antigens can be isolated and purified as described herein. Methods for preparing and isolating polyclonal and monoclonal antibodies are well known in the art. See, for example, Current Protocols in Immunology, Cooligan, et al. (eds.), National Institutes of Health, John Wiley and Sons, Inc., 1995; Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Inc., Boca. Raton, Fla., 1982.

As would be evident to one of ordinary skill in the art, polyclonal antibodies can be generated from inoculating a variety of warm-blooded animals such as horses, cows, goats, sheep, dogs, chickens, rabbits, mice, and rats with a zalpha48 or zsig97 polypeptide or a fragment thereof. The immunogenicity of a zalpha48 or zsig97 polypeptide may be increased through the use of an adjuvant, such as alum (aluminum hydroxide) or Freund's complete or incomplete adjuvant. Polypeptides useful for immunization also include fusion polypeptides, such as fusions of zalpha48 or zsig97 or a portion thereof with an immunoglobulin polypeptide or with maltose binding protein. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

As used herein, the term “antibodies” includes polyclonal antibodies, affinity-purified polyclonal antibodies, monoclonal antibodies, and antigen-binding fragments, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced.

Alternative techniques for generating or selecting antibodies useful herein include in vitro exposure of lymphocytes to zalpha48 or zsig97 protein or peptide, and selection of antibody display libraries in phage or similar vectors (for instance, through use of immobilized or labeled zalpha48 or zsig97 protein or peptide). Genes encoding polypeptides having potential zalpha48 or zsig97 polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides which interact with a known target which can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially, for instance from Clontech (Palo Alto, Calif.), Invitrogen Inc. (San Diego, Calif.), New England Biolabs, Inc. (Beverly, Mass.) and Pharmacia LKB Biotechnology Inc. (Piscataway, N.J.). Random peptide display libraries can be screened using the zalpha48 or zsig97 sequences disclosed herein to identify proteins which bind to zalpha48 or zsig97. These “binding polypeptides” which interact with zalpha48 or zsig97 polypeptides can be used for tagging tissues or cells, such as the specific tissues or cells in which zalpha48 or zsig97 is expressed, e.g., pituitary, testis, and spleen; for isolating homolog polypeptides by affinity purification; they can be directly or indirectly conjugated to drugs, toxins, radionuclides and the like. These binding polypeptides can also be used in analytical methods such as for screening expression libraries and neutralizing activity, e.g., for blocking interaction between ligand and receptor, or viral binding to a receptor. The binding polypeptides can also be used for diagnostic assays for determining circulating levels of zalpha48 or zsig97 polypeptides; for detecting or quantitating soluble zalpha48 or zsig97 polypeptides as marker of underlying pathology or disease. These binding polypeptides can also act as zalpha48 or zsig97 “antagonists” to block zalpha48 or zsig97 binding and signal transduction in vitro and in vivo. These anti-zalpha48 or zsig97 binding polypeptides would be useful for inhibiting zalpha48 or zsig97 activity or protein-binding.

Antibodies are considered to be specifically binding if: 1) they exhibit a threshold level of binding activity, and 2) they do not significantly cross-react with related polypeptide molecules. A threshold level of binding is determined if anti-zalpha48 or zsig97 antibodies herein bind to a zalpha48 or zsig97 polypeptide, peptide or epitope with an affinity at least 10-fold greater than the binding affinity to control (non-zalpha48 or zsig97) polypeptide. It is preferred that the antibodies exhibit a binding affinity (Kₐ) of 10⁶ M⁻¹ or greater, preferably 10⁷ M⁻¹ or greater, more preferably 10⁸ M⁻¹ or greater, and most preferably 10 M⁻¹ or greater. The binding affinity of an antibody can be readily determined by one of ordinary skill in the art, for example, by Scatchard analysis (Scatchard, G., Ann. NY Acad. Sci. 51: 660-672, 1949).

Whether anti-zalpha48 or zsig97 antibodies do not significantly cross-react with related polypeptide molecules is shown, for example, by the antibody detecting zalpha48 or zsig97 polypeptide but not known related polypeptides using a standard Western blot analysis (Ausubel et al., ibid.). Examples of known related polypeptides are those disclosed in the prior art, such as known orthologs, and paralogs, and similar known members of a protein family, Screening can also be done using non-human zalpha48 or zsig97, and zalpha48 or zsig97 mutant polypeptides. Moreover, antibodies can be “screened against” known related polypeptides, to isolate a population that specifically binds to the zalpha48 or zsig97 polypeptides. For example, antibodies raised to zalpha48 or zsig97 are adsorbed to related polypeptides adhered to insoluble matrix; antibodies specific to zalpha48 or zsig97 will flow through the matrix under the proper buffer conditions. Screening allows isolation of polyclonal and monoclonal antibodies non-crossreactive to known closely related polypeptides (Antibodies: A Laboratory Manual, Harlow and Lane (eds.), Cold Spring Harbor Laboratory Press, 1988; Current Protocols in Immunology, Cooligan, et al. (eds.), National Institutes of Health, John Wiley and Sons, Inc., 1995). Screening and isolation of specific antibodies is well known in the art. See, Fundamental Immunology, Paul (eds.), Raven Press, 1993; Getzoff et al., Adv. in Immunol. 43: 1-98, 1988; Monoclonal Antibodies: Principles and Practice, Goding, J. W. (eds.), Academic Press Ltd., 1996; Benjamin et al., Ann. Rev. Immunol. 2: 67-101, 1984. Specifically binding anti-zalpha48 or zsig97 antibodies can be detected by a number of methods in the art, and disclosed below.

A variety of assays known to those skilled in the art can be utilized to detect antibodies which bind to zalpha48 or zsig97 proteins or polypeptides. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radioimmunoassay, radioimmuno-precipitation, enzyme-linked immunosorbent assay (ELISA), dot blot or Western blot assay, inhibition or competition assay, and sandwich assay. In addition, antibodies can be screened for binding to wild-type versus mutant zalpha48 or zsig97 protein or polypeptide. Antibodies to zalpha48 or zsig97 may be used for tagging cells or tissues that express zalpha48 or zsig97, e.g., testis, pituitary, and spleen cells or tissues; for isolating zalpha48 or zsig97 by affinity purification; for diagnostic assays for determining circulating levels of zalpha48 or zsig97 polypeptides; for detecting or quantitating soluble zalpha48 or zsig97 as marker of underlying pathology or disease; in analytical methods employing FACS; for screening expression libraries; for generating anti-idiotypic antibodies; and as neutralizing antibodies or as antagonists to block zalpha48 or zsig97 activity in vitro and in vivo. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates. Antibodies herein may also be directly or indirectly conjugated to drugs, toxins, radionuclides and the like, and these conjugates used for in vivo diagnostic or therapeutic applications. Moreover, antibodies to zalpha48 or zsig97 or fragments thereof may be used in vitro to detect denatured zalpha48 or zsig97 or fragments thereof in assays, for example, Western Blots or other assays known in the art.

Antibodies or polypeptides herein can also be directly or indirectly conjugated to drugs, toxins, radionuclides and the like, and these conjugates used for in vivo diagnostic or therapeutic applications. For instance, polypeptides or antibodies of the present invention can be used to identify or treat tissues or organs that express a corresponding anti-complementary molecule (receptor or antigen, respectively, for instance). More specifically, zalpha48 or zsig97 polypeptides or anti-zalpha48 or zsig97 antibodies, or bioactive fragments or portions thereof, can be coupled to detectable or cytotoxic molecules and delivered to a mammal having cells, tissues or organs that express the anti-complementary molecule.

Suitable detectable molecules may be directly or indirectly attached to the polypeptide or antibody, and include radionuclides, enzymes, substrates, cofactors, inhibitors, fluorescent markers, chemiluminescent markers, magnetic particles and the like. Suitable cytotoxic molecules may be directly or indirectly attached to the polypeptide or antibody, and include bacterial or plant toxins (for instance, diphtheria toxin, Pseudomonas exotoxin, ricin, abrin and the like), as well as therapeutic radionuclides, such as iodine-131, rhenium-188 or yttrium-90 (either directly attached to the polypeptide or antibody, or indirectly attached through means of a chelating moiety, for instance). Polypeptides or antibodies may also be conjugated to cytotoxic drugs, such as adriamycin. For indirect attachment of a detectable or cytotoxic molecule, the detectable or cytotoxic molecule can be conjugated with a member of a complementary/anticomplementary pair, where the other member is bound to the polypeptide or antibody portion. For these purposes, biotin/streptavidin is an exemplary complementary/anticomplementary pair.

In another embodiment, polypeptide-toxin fusion proteins or antibody-toxin fusion proteins can be used for targeted cell or tissue inhibition or ablation (for instance, to treat cancer cells or tissues). Alternatively, if the polypeptide has multiple functional domains (i.e., an activation domain or a receptior binding domain, plus a targeting domain), a fusion protein including only the targeting domain may be suitable for directing a detectable molecule, a cytotoxic molecule or a complementary molecule to a cell or tissue type of interest. In instances where the domain only fusion protein includes a complementary molecule, the anti-complementary molecule can be conjugated to a detectable or cytotoxic molecule. Such domain-complementary molecule fusion proteins thus represent a generic targeting vehicle for cell/tissue-specific delivery of generic anti-complementary-detectable/cytotoxic molecule conjugates.

In another embodiment, zalpha48 or zsig97-cytokine fusion proteins or antibody-cytokine fusion proteins can be used for enhancing in vivo killing of target tissues (for example, blood and bone marrow cancers), if the zalpha48 or zsig97 polypeptide or anti-zalpha48 or zsig97 antibody targets the hyperproliferative blood or bone marrow cell (See, generally, Hornick et al., Blood 89:4437-47, 1997). Hornick et al. described fusion proteins that target a cytokine to a desired site of action, thereby providing an elevated local concentration of cytokine. Suitable zalpha48 or zsig97 polypeptides or anti-zalpha48 or zsig97 antibodies can target an undesirable cell or tissue (i.e., a tumor or a leukemia), and the fused cytokine can mediate improved target cell lysis by effector cells. Suitable cytokines for this purpose include interleukin 2 and granulocyte-macrophage colony-stimulating factor (GM-CSF), for instance.

In yet another embodiment, if the zalpha48 or zsig97 polypeptide or anti-zalpha48 or zsig97 antibody targets vascular cells or tissues, such polypeptide or antibody may be conjugated with a radionuclide, and particularly with a beta-emitting radionuclide, to reduce restenosis. Such therapeutic approach poses less danger to clinicians who administer the radioactive therapy. For instance, iridium-192 impregnated ribbons placed into stented vessels of patients until the required radiation dose was delivered showed decreased tissue growth in the vessel and greater luminal diameter than the control group, which received placebo ribbons. Further, revascularisation and stent thrombosis were significantly lower in the treatment group. Similar results are predicted with targeting of a bioactive conjugate containing a radionuclide, as described herein.

The bioactive polypeptide or antibody conjugates described herein can be delivered intravenously, intraarterially or intraductally, or may be introduced locally at the intended site of action.

Molecules of the present invention can be used to identify and isolate receptors that bind zalpha48 or zsig97 polypeptide. For example, proteins and peptides of the present invention can be immobilized on a column and membrane preparations run over the column (Immobilized Affinity Ligand Techniques, Hermanson et al., eds., Academic Press, San Diego, Calif., 1992, pp. 195-202). Proteins and peptides can also be radiolabeled (Methods in Enzymol., vol. 182, “Guide to Protein Purification”, M. Deutscher, ed., Acad. Press, San Diego, 1990, 721-37) or photoaffinity labeled (Brunner et al., Ann. Rev. Biochem. 62:483-514, 1993 and Fedan et al., Biochem. Pharmacol. 33:1167-80, 1984) and specific cell-surface proteins can be identified. One specific method of isolating the receptors of the mature peptides of the present invention would involve [insert method described in journal article here.]

The polypeptides, antagonists, agonists, nucleic acid and/or antibodies of the present invention may be used in diagnosis and treatment of disorders associated with gonadal development, pregnancy, pubertal changes, menopause, ovarian cancer, fertility, ovarian function, polycystic ovarian syndrome, uterine cancer, endometriosis, libido, mylagia and neuralgia associated with reproductive phenomena, prostate cancer, thyroid disease, adrenal dysfunction, pituitary disfunction, and cancers of the immune system such as acute myocytic leukemia. The molecules of the present invention may used to modulate or to treat or prevent development of pathological conditions in such diverse tissue as prostate and the pancreas. In particular, certain syndromes or diseases may be amenable to such diagnosis, treatment or prevention.

Zalpha48 or zsig97 polypeptide may have additional biological activity in the female reproductive system independent of prostate function, as described herein. Oogenesis is the process by which a diploid stem cell proceeds through multiple stages of differentiation, culminating in the formation of a terminally differentiated cell with a unique function, an oocyte. Unlike spermatogenesis, which begins at puberty and continues on through the life of a male, oogenesis begins during fetal development and by birth, a female's entire supply of primary oocytes are stored in the ovaries in primordial follicles and await maturation and release.

In the adult ovary, folliculogenesis starts when the follicles enter the growth phase. Early growing follicles undergo a dramatic process of cellular proliferation and differentiation. The classic control of ovarian function by luteinizing hormone (LH) and follicle stimulating hormone (FSH) is now thought to include the action of a variety of molecules that act to promote cell-cell interactions between cells of the follicle. For review, see Gougeon, A., Endocrine Rev. 17:121-155, 1996. Hence, the mechanisms for controlling ovarian folliculogenesis and dominant follicle selection are still under investigation. As zalpha48 or zsig97 is expressed in the uterus, it may serve a role in modulating ovarian function by regulating folliculogenesis and dominant follicle selection, by affecting proliferation or differentiation of follicular cells, affecting cell-cell interactions, modulating hormones involved in the process, and the like.

The ovarian cycle in mammals includes the growth and maturation of follicles, followed by ovulation and transformation of follicles into corpea lutea. The physiological events in the ovarian cycle are dependent on interactions between hormones and cells within the hypothalamic-pituitary-ovarian axis, including gonadotropin releasing hormone (GnRH), LH, and FSH. In addition, estradiol, synthesized in the follicle, primes the hypothalamic-pituitary axis and is required for the mid-cycle surge of gonadotropin that stimulates the resumption of oocyte meiosis and leads to ovulation and subsequent extrusion of an oocyte from the follicle. This gonadotropin surge also promotes the differentiation of the follicular cells from secreting estradiol to secreting progesterone. Progesterone, secreted by the corpus luteum, is needed for uterine development required for the implantation of fertilized oocytes. The central role of hypothalamic-pituitary-gonadal hormones in the ovarian cycle and reproductive cascade, and the role of sex steroids on target tissues and organs, e.g., uterus, breast, adipose, bones and liver, has made modulators of their activity desirable for therapeutic applications. Such applications include treatments for precocious puberty, endometriosis, uterine leiomyomata, hirsutism, infertility, pre menstrual syndrome (PMS), amenorrhea, and as contraceptive agents.

Zalpha48 or zsig97 polypeptides, agonists and antagonists which modulate the actions of such hormones can be of therapeutic value. Such molecules can also be useful for modulating steroidogenesis, both in vivo and in vitro, and modulating aspects of the ovarian cycle such as oocyte maturation, ovarian cell-cell interactions, follicular development and rupture, luteal function, menstruation, and promoting uterine implantation of fertilized oocytes. Molecules which modulate hormone action can be beneficial therapeutics for use prior to or at onset of puberty, or in adult women.

For example, puberty in females is marked by an establishment of feed-back loops to control hormone levels and hormone production. Abnormalities resulting from hormone imbalances during puberty have been observed and include precocious puberty, where pubertal changes occur in females prior to the age of 8. Hormone-modulating molecules, can be used, in this case, to suppress hormone secretion and delay onset of puberty.

The level and ratio of gonadotropin and steroid hormones can be used to assess the existence of hormonal imbalances associated with diseases, as well as determine whether normal hormonal balance has been restored after administration of a therapeutic agent. Determination of estradiol, progesterone, LH, and FSH, for example, from serum is known by one of skill in the art. Such assays can be used to monitor the hormone levels after administration of zalpha48 or zsig97 in vivo, or in a transgenic mouse model where the zalpha48 or zsig97 gene is expressed or the murine ortholog is deleted. Thus, as a hormone-modulating molecule, zalpha48 or zsig97 polypeptides can have therapeutic application for treating, for example, breakthrough menopausal bleeding, as part of a therapeutic regime for pregnancy support, or for treating symptoms associated with polycystic ovarian syndrome (PCOS), endometriosis, PMS and menopause. In addition, other in vivo rodent models are known in the art to assay effects of zalpha48 or zsig97 polypeptide on, for example, polycystic ovarian syndrome (PCOS).

Proteins of the present invention may also be used in applications for enhancing fertilization during assisted reproduction in humans and in animals. Such assisted reproduction methods are known in the art and include artificial insemination, in vitro fertilization, embryo transfer, and gamete intrafallopian transfer. Such methods are useful for assisting those who may have physiological or metabolic disorders that prevent or impede natural conception. Such methods are also used in animal breeding programs, e.g., for livestock, racehorses, domestic and wild animals, and could be used as methods for the creation of transgenic animals. Zalpha48 or zsig97 polypeptides could be used in the induction of ovulation, either independently or in conjunction with a regimen of gonadotropins or agents such as clomiphene citrate or bromocriptine (Speroff et al., Induction of ovulation, Clinical Gynecolopic Endocrinology and Infertility, 5^{th} ed., Baltimore, Williams & Wilkins, 1994). As such, proteins of the present invention can be administered to the recipient prior to fertilization or combined with the sperm, an egg or an egg-sperm mixture prior to in vitro or in vivo fertilization. Such proteins can also be mixed with oocytes prior to cryopreservation to enhance viability of the preserved oocytes for use in assisted reproduction.

The zalpha48 or zsig97 polypeptides, agonists and antagonists of the present invention may be directly used as or incorporated into therapies for treating reproductive disorders. Disorders such as luteal phase deficiency would benefit from such therapy (Soules, “Luteal phase deficiency: A subtle abnormality of ovulation” in, Infertility: Evaluation and Treatment, Keye et al., eds., Philadelphia, W B Saunders, 1995). Moreover, administration of gonadotropin-releasing hormone is shown to stimulate reproductive behavior (Riskin and Moss, Res. Bull. 11:481-5, 1983; Kadar et al., Physiol. Behav. 51:601-5, 1992 and Silver et al., J. Neruoendocrin. 4:207-10, 199; King and Millar, Cell. Mol. Neurobiol., 15:5-23, 1995). Given the high prevalence of sexual dysfunction and impotence in humans, molecules, such as zalpha48 or zsig97, which may modulate or enhance gonadotropin activity can find application in developing treatments for these conditions. Conversely, polypeptides of the present invention, their antagonists or agonists can be used to inhibit normal reproduction in the form of birth control, for example, by decreasing spermatogenesis or preventing uterine implantation of a fertilized egg.

The zalpha48 or zsig97 polypeptides of the present invention can be used to study ovarian cell proliferation, maturation, and differentiation, i.e., by acting as a luteinizing agent that converts granulosa cells from estradiol to progesterone-producing cells. Such methods of the present invention generally comprise incubating granulosa cells, theca cells, oocytes or a combination thereof, in the presence and absence of zalpha48 or zsig97 polypeptide, monoclonal antibody, agonist or antagonist thereof and observing changes in cell proliferation, maturation and differentiation. See for example, Basini et al., (J. Rep. Immunol. 37:139-53, 1998); Duleba et al., (Fert. Ster. 69:335-40, 1998); and Campbell, B. K. et al., J. Reprod. and Fert. 112:69-77, 1998).

The motor and neurological affects of molecules of the present invention make it useful for treatment of obesity and other metabolic disorders where neurological feedback modulates nutritional absorption. The molecules of the present invention are useful for regulating satiety, glucose absorption and metabolism, and neuropathy-associated gastrointestinal disorders. Molecules of the present invention are also useful as additives to anti-hypoglycemic preparations containing glucose and as adsorption enhancers for oral drugs which require fast nutrient action. Additionally, molecules of the present invention can be used to stimulate glucose-induced insulin release.

Moreover, tissues in which the polypeptides of the present invention may be expressed are comprised in part of epithelial cells where zalpha48 or zsig97 polypeptides, agonists or antagonists thereof may be therapeutically useful for promoting wound healing. To verify the presence of this capability in zalpha48 or zsig97 polypeptides, agonists or antagonists of the present invention, such zalpha48 or zsig97 polypeptides, agonists or antagonists are evaluated with respect to their ability to facilitate wound healing according to procedures known in the art. If desired, zalpha48 or zsig97 polypeptide performance in this regard can be compared to growth factors, such as EGF, NGF, TGF-α, TGF-β, insulin, IGF-I, IGF-II, fibroblast growth factor (FGF) and the like. Moreover, the effects of zalpha48 or zsig97 polypeptides, agonists or antagonists thereof can be evaluated with respect to their ability to enhance wound contractility involved in wound healing. In addition, zalpha48 or zsig97 polypeptides or agonists or antagonists thereof may be evaluated in combination with one or more growth factors to identify synergistic effects.

The molecules of the present invention are useful as components of defined cell culture media, as described herein, and may be used alone or in combination with other cytokines and hormones to replace serum that is commonly used in cell culture. Molecules of the present invention are particularly useful in specifically promoting the growth, development, differentiation, and/or maturation of ovarian cells in culture, and may also prove useful in the study of the ovarian cycle, reproductive function, ovarian and testicular cell-cell interactions, sperm capacitation and fertilization.

In addition, the present invention also provides methods for studying steroidogenesis and steroid hormone secretion. Such methods generally comprise incubating ovarian cells in culture medium comprising zalpha48 or zsig97 polypeptides, monoclonal antibodies, agonists or antagonists thereof with and without gonadotropins and/or steroid hormones, and subsequently observing protein and steroid secretion. Exemplary gonadotropin hormones include luteinizing hormone and follicle stimulating hormone (Rouillier et al., Mol. Reprod. Dev. 50:170-7, 1998). Exemplary steroid hormones include estradiol, androstenedione, and progesterone. Effects of zalpha48 or zsig97 on steroidogenesis or steroid secretion can be determined by methods known in the art, such as radioimmunoassay (to detect levels of estradiol, androstenedione, progesterone, and the like), and immunoradiometric assay (IRMA).

Molecules that are cleaved into smaller bioactive peptides, such as zalpha48 or zsig97 polypeptide, can modulate hormones, hormone receptors, growth factors, or cell-cell interactions, of the reproductive cascade or are involved in oocyte or ovarian development, or the like, would be useful as markers for cancer of reproductive organs and other tissues, and as therapeutic agents for hormone-dependent cancers, by inhibiting hormone-dependent growth and/or development of tumor cells. Human reproductive system cancers such as ovarian, uterine, cervical, testicular and prostate cancers are common. Moreover, receptors for steroid hormones involved in the reproductive cascade are found in human tumors and tumor cell lines (breast, prostate, endometrial, ovarian, kidney, and pancreatic tumors) (Kakar et al., Mol. Cell. Endocrinol., 106:145-49, 1994; Kakar and Jennes, Cancer Letts., 98:57-62, 1995). Thus, expression of zalpha48 or zsig97 in reproductive tissues suggests that polypeptides of the present invention would be useful in diagnostic methods for the detection and monitoring of reproductive and gastric cancers, as well as other cancers.

Diagnostic methods of the present invention involve the detection of zalpha48 or zsig97 polypeptides in the serum or tissue biopsy of a patient undergoing analysis of reproductive function or evaluation for possible reproductive cancers, e.g., ovarian or prostate cancer. Such polypeptides can be detected using immunoassay techniques and antibodies, described herein, that are capable of recognizing zalpha48 or zsig97 polypeptide epitopes. More specifically, the present invention contemplates methods for detecting zalpha48 or zsig97 polypeptides comprising:
exposing a test sample potentially containing zalpha48 or zsig97 polypeptides to an antibody attached to a solid support, wherein said antibody binds to a first epitope of a zalpha48 or zsig97 polypeptide;
washing the immobilized antibody-polypeptide to remove unbound contaminants;
exposing the immobilized antibody-polypeptide to a second antibody directed to a second epitope of a zalpha48 or zsig97 polypeptide, wherein the second antibody is associated with a detectable label; and
detecting the detectable label. Altered levels of zalpha48 or zsig97 polypeptides in a test sample, such as serum sweat, saliva, biopsy, and the like, can be monitored as an indication of reproductive function or of reproductive cancer or disease, when compared against a normal control. Similarly, such methods can be used to detect the presence of tissues in which zalpha48 or zsig97 is expressed, such as testis, pituitary, and spleen tissues. In comparison to a control, the detection of testis, pituitary, and spleen disease, such as cancer, inflammation, or other dysfunction can be achieved using the polynucleotides, polypeptides, or antibodies of the present invention.

Additional methods using probes or primers derived, for example, from the nucleotide sequences disclosed herein can also be used to detect zalpha48 or zsig97 expression in a patient sample, such as a blood, saliva, sweat, biopsy, tissue sample, or the like. For example, probes can be hybridized to tumor tissues and the hybridized complex detected by in situ hybridization. Zalpha48 or zsig97 sequences can also be detected by PCR amplification using cDNA generated by reverse translation of sample mRNA as a template (PCR Primer A Laboratory Manual, Dieffenbach and Dveksler, eds., Cold Spring Harbor Press, 1995). When compared with a normal control, both increases or decreases of zalpha48 or zsig97 expression in a patient sample, relative to that of a control, can be monitored and used as an indicator or diagnostic for disease. For example, such methods can be used to detect the presence of tissues in which zalpha48 or zsig97 is expressed, such as ovary, pituitary, and pancreatic tissues. In comparison to a control, the detection of thyroid, ovary, pituitary, and pancreatic disease, such as cancer, inflammation, or other dysfunction can be achieved using the polynucleotides, polypeptides, or antibodies of the present invention.

Differentiation is a progressive and dynamic process, beginning with pluripotent stem cells and ending with terminally differentiated cells. Pluripotent stem cells that can regenerate without commitment to a lineage express a set of differentiation markers that are lost when commitment to a cell lineage is made.

Progenitor cells express a set of differentiation markers that may or may not continue to be expressed as the cells progress down the cell lineage pathway toward maturation.

Differentiation markers that are expressed exclusively by mature cells are usually functional properties such as cell products, enzymes to produce cell products, and receptors. The stage of a cell population's differentiation is monitored by identification of markers present in the cell population. The novel polypeptides of the present invention may be useful for studies to isolate stem cells and neuronal or other progenitor cells, both in vivo and ex vivo.

There is evidence to suggest that factors that stimulate specific cell types down a pathway towards terminal differentiation or dedifferentiation affect the entire cell population originating from a common precursor or stem cell. Assays measuring differentiation include, for example, measuring cell markers associated with stage-specific expression of a tissue, enzymatic activity, functional activity or morphological changes (Watt, FASEB, 5:281-284, 1991; Francis, Differentiation 57:63-75, 1994; Raes, Adv. Anim. Cell Biol. Technol. Bioprocesses, 161-171, 1989; all incorporated herein by reference). Alternatively, zalpha48 or zsig97 polypeptide itself can serve as an additional cell-surface or secreted marker associated with stage-specific expression of a tissue, such as testis tissue. As such, direct measurement of zalpha48 or zsig97 polypeptide, or its loss of expression in a tissue as it differentiates, can serve as a marker for differentiation of tissues.

Similarly, direct measurement of zalpha48 or zsig97 polypeptide, or its loss of expression in a tissue can be determined in a tissue or cells as they undergo tumor or disease progression. Increases in invasiveness and motility of cells, or the gain or loss of expression of zalpha48 or zsig97 in a pre-cancerous or cancerous condition, in comparison to normal tissue, can serve as a diagnostic for transformation, invasion and metastasis in tumor progression. As such, knowledge of a tumor's stage of progression or metastasis will aid the physician in choosing the most proper therapy, or aggressiveness of treatment, for a given individual cancer patient. Methods of measuring gain and loss of expression (of either mRNA or protein) are well known in the art and described herein and can be applied to zalpha48 or zsig97 expression. For example, appearance or disappearance of polypeptides that regulate cell motility can be used to aid diagnosis and prognosis of prostate cancer (Banyard, J. and Zetter, B. R., Cancer and Metast. Rev. 17:449-458, 1999). As as a thyroid, pituitary, and pancreatic-specific marker, zalpha48 or zsig97 gain or loss of expression may serve as a diagnostic for thyroid, pituitary, and pancreatic tumor tissue, and other cancers. Moreover, analogous to the prostate specific antigen (PSA), as a naturally-expressed thyroid, pituitary, and pancreatic marker, increased levels of zalpha48 or zsig97 polypeptides, or anti-zalpha48 or zsig97 antibodies in a patient, relative to a normal control can be indicative of thyroid, prostate, pancreatic, adrenal or ovarian cancer (See, e.g., Mulders, TMT, et al., Eur. J. Surgical Oncol. 16:37-41, 1990). Moreover, as zalpha48 or zsig97 expression appears to be restricted to specific human tissues, lack of zalpha48 or zsig97 expression in those tissues or strong zalpha48 or zsig97 expression in tissues where zalpha48 or zsig97 is not normally expressed, would serve as a diagnostic of an abnormality in the cell or tissue type, of invasion or metastasis of cancerous testis, pituitary, and spleen tissues into non-testis, pituitary, and spleen tissue, and could aid a physician in directing further testing or investigation, or aid in directing therapy.

Polynucleotides encoding zalpha48 or zsig97 polypeptides are useful within gene therapy or gene transfer applications where it is desired to increase or inhibit zalpha48 or zsig97 activity. If a mammal has a mutated or absent zalpha48 or zsig97 gene, the zalpha48 or zsig97 gene can be introduced into the cells of the mammal. In one embodiment, a gene encoding a zalpha48 or zsig97 polypeptide is introduced in vivo in a viral vector. Such vectors include an attenuated or defective DNA virus, such as, but not limited to, herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes simplex virus 1 (HSV1) vector (Kaplitt et al., Molec. Cell. Neurosci. 2:320-30, 1991); an attenuated adenovirus vector, such as the vector described by Stratford-Perricaudet et al., J. Clin. Invest. 90:626-30, 1992; and a defective adeno-associated virus vector (Samulski et al., J. Virol. 61:3096-101, 1987; Samulski et al., J. Virol. 63:3822-8, 1989).

In another embodiment, a zalpha48 or zsig97 gene can be introduced in a retroviral vector, e.g., as described in Anderson et al., U.S. Pat. No. 5,399,346; Mann et al. Cell 33:153, 1983; Temin et al., U.S. Pat. No. 4,650,764; Temin et al., U.S. Pat. No. 4,980,289; Markowitz et al., J. Virol. 62:1120, 1988; Temin et al., U.S. Pat. No. 5,124,263; International Patent Publication No. WO 95/07358, published Mar. 16, 1995 by Dougherty et al.; and Kuo et al., Blood 82:845, 1993. Alternatively, the vector can be introduced by lipofection in vivo using liposomes. Synthetic cationic lipids can be used to prepare liposomes for in vivo transfection of a gene encoding a marker (Felgner et al., Proc. Natl. Acad. Sci. USA 84:7413-7, 1987; Mackey et al., Proc. Natl. Acad. Sci. USA 85:8027-31, 1988). The use of lipofection to introduce exogenous genes into specific organs in vivo has certain practical advantages. Molecular targeting of liposomes to specific cells represents one area of benefit. More particularly, directing transfection to particular cells represents one area of benefit. For instance, directing transfection to particular cell types would be particularly advantageous in a tissue with cellular heterogeneity, such as the pancreas, liver, kidney, and brain. Lipids may be chemically coupled to other molecules for the purpose of targeting. Targeted peptides (e.g., hormones or neurotransmitters), proteins such as antibodies, or non-peptide molecules can be coupled to liposomes chemically.

It is possible to remove the target cells from the body; to introduce the vector as a naked DNA plasmid; and then to re-implant the transformed cells into the body. Naked DNA vectors for gene therapy or gene transfer can be introduced into the desired host cells by methods known in the art, e.g., transfection, electroporation, microinjection, transduction, cell fusion, DEAE dextran, calcium phosphate precipitation, use of a gene gun or use of a DNA vector transporter. See, e.g., Wu et al., J. Biol. Chem. 267:963-7, 1992; Wu et al., J. Biol. Chem. 263:14621-4, 1988.

Antisense methodology can be used to inhibit zalpha48 or zsig97 gene transcription, such as to inhibit cell proliferation in vivo. Polynucleotides that are complementary to a segment of a zalpha48 or zsig97-encoding polynucleotide (e.g., a polynucleotide as set froth in SEQ ID NO:1) are designed to bind to zalpha48 or zsig97-encoding mRNA and to inhibit translation of such mRNA. Such antisense polynucleotides are used to inhibit expression of zalpha48 or zsig97 polypeptide-encoding genes in cell culture or in a subject.

Defects in the zalpha48 or zsig97 locus itself may result in a heritable human disease state. Molecules of the present invention, such as the polypeptides, antagonists, agonists, polynucleotides and antibodies of the present invention would aid in the detection, diagnosis prevention, and treatment associated with a zalpha48 or zsig97 genetic defect.

A diagnostic could assist physicians in determining the type of disease and appropriate associated therapy, or assistance in genetic counseling. As such, the inventive anti-zalpha48 or zsig97 antibodies, polynucleotides, and polypeptides can be used for the detection of zalpha48 or zsig97 polypeptide, mRNA or anti-zalpha48 or zsig97 antibodies, thus serving as markers and be directly used for detecting or genetic diseases or cancers, as described herein, using methods known in the art and described herein. Molecules of the present invention, such as the polypeptides, antagonists, agonists, polynucleotides and antibodies of the present invention would aid in the detection, diagnosis prevention, and treatment associated with a zalpha48 or zsig97 genetic defect. In addition, zalpha48 or zsig97 polynucleotide probes can be used to detect allelic differences between diseased or non-diseased individuals at the zalpha48 or zsig97 chromosomal locus. As such, the zalpha48 or zsig97 sequences can be used as diagnostics in forensic DNA profiling.

In general, the diagnostic methods used in genetic linkage analysis, to detect a genetic abnormality or aberration in a patient, are known in the art. Analytical probes will be generally at least 20 nt in length, although somewhat shorter probes can be used (e.g., 14-17 nt). PCR primers are at least 5 nt in length, preferably 15 or more, more preferably 20-30 nt. For gross analysis of genes, or chromosomal DNA, a zalpha48 or zsig97 polynucleotide probe may comprise an entire exon or more. Exons are readily determined by one of skill in the art by comparing zalpha48 or zsig97 sequences (SEQ ID NO:1) with the human genomic DNA for zalpha48 or zsig97 (Entrez Accession No. AC006116). In general, the diagnostic methods used in genetic linkage analysis, to detect a genetic abnormality or aberration in a patient, are known in the art. Most diagnostic methods comprise the steps of (a) obtaining a genetic sample from a potentially diseased patient, diseased patient or potential non-diseased carrier of a recessive disease allele; (b) producing a first reaction product by incubating the genetic sample with a ZSMF16 polynucleotide probe wherein the polynucleotide will hybridize to complementary polynucleotide sequence, such as in RFLP analysis or by incubating the genetic sample with sense and antisense primers in a PCR reaction under appropriate PCR reaction conditions; (iii) Visualizing the first reaction product by gel electrophoresis and/or other known method such as visualizing the first reaction product with a ZSMF16 polynucleotide probe wherein the polynucleotide will hybridize to the complementary polynucleotide sequence of the first reaction; and (iv) comparing the visualized first reaction product to a second control reaction product of a genetic sample from wild type patient. A difference between the first reaction product and the control reaction product is indicative of a genetic abnormality in the diseased or potentially diseased patient, or the presence of a heterozygous recessive carrier phenotype for a non-diseased patient, or the presence of a genetic defect in a tumor from a diseased patient, or the presence of a genetic abnormality in a fetus or pre-implantation embryo. For example, a difference in restriction fragment pattern, length of PCR products, length of repetitive sequences at the zalpha48 or zsig97 genetic locus, and the like, are indicative of a genetic abnormality, genetic aberration, or allelic difference in comparison to the normal wild type control. Controls can be from unaffected family members, or unrelated individuals, depending on the test and availability of samples. Genetic samples for use within the present invention include genomic DNA, mRNA, and cDNA isolated form any tissue or other biological sample from a patient, such as but not limited to, blood, saliva, semen, embryonic cells, amniotic fluid, and the like. The polynucleotide probe or primer can be RNA or DNA, and will comprise a portion of SEQ ID NO:1, the complement of SEQ ID NO:1, or an RNA equivalent thereof. Such methods of showing genetic linkage analysis to human disease phenotypes are well known in the art. For reference to PCR based methods in diagnostics see see, generally, Mathew (ed.), Protocols in Human Molecular Genetics (Humana Press, Inc. 1991), White (ed.), PCR Protocols: Current Methods and Applications (Humana Press, Inc. 1993), Cotter (ed.), Molecular Diagnosis of Cancer (Humana Press, Inc. 1996), Hanausek and Walaszek (eds.), Tumor Marker Protocols (Humana Press, Inc. 1998), Lo (ed.), Clinical Applications of PCR (Humana Press, Inc. 1998), and Meltzer (ed.), PCR in Bioanalysis (Humana Press, Inc. 1998)).

Aberrations associated with the zalpha48 or zsig97 locus can be detected using nucleic acid molecules of the present invention by employing standard methods for direct mutation analysis, such as restriction fragment length polymorphism analysis, short tandem repeat analysis employing PCR techniques, amplification-refractory mutation system analysis, single-strand conformation polymorphism detection, RNase cleavage methods, denaturing gradient gel electrophoresis, fluorescence-assisted mismatch analysis, and other genetic analysis techniques known in the art (see, for example, Mathew (ed.), Protocols in Human Molecular Genetics (Humana Press, Inc. 1991), Marian, Chest 108:255 (1995), Coleman and Tsongalis, Molecular Diagnostics (Human Press, Inc. 1996), Elles (ed.) Molecular Diagnosis of Genetic Diseases (Humana Press, Inc. 1996), Landegren (ed.), Laboratory Protocols for Mutation Detection (Oxford University Press 1996), Birren et al. (eds.), Genome Analysis, Vol. 2: Detecting Genes (Cold Spring Harbor Laboratory Press 1998), Dracopoli et al. (eds.), Current Protocols in Human Genetics (John Wiley & Sons 1998), and Richards and Ward, “Molecular Diagnostic Testing,” in Principles of Molecular Medicine, pages 83-88 (Humana Press, Inc. 1998)). Direct analysis of an zalpha48 or zsig97 gene for a mutation can be performed using a subject's genomic DNA. Methods for amplifying genomic DNA, obtained for example from peripheral blood lymphocytes, are well-known to those of skill in the art (see, for example, Dracopoli et al. (eds.), Current Protocols in Human Genetics, at pages 7.1.6 to 7.1.7 (John Wiley & Sons 1998)).

Mice engineered to express the zalpha48 or zsig97 gene, referred to as “transgenic mice,” and mice that exhibit a complete absence of zalpha48 or zsig97 gene function, referred to as “knockout mice,” may also be generated (Snouwaert et al., Science 257:1083, 1992; Lowell et al., Nature 366:740-42, 1993; Capecchi, M. R., Science 244: 1288-1292, 1989; Palmiter, R. D. et al. Annu Rev Genet. 20: 465-499, 1986). For example, transgenic mice that over-express zalpha48 or zsig97, either ubiquitously or under a tissue-specific or tissue-restricted promoter can be used to ask whether over-expression causes a phenotype. For example, over-expression of a wild-type zalpha48 or zsig97 polypeptide, polypeptide fragment or a mutant thereof may alter normal cellular processes, resulting in a phenotype that identifies a tissue in which zalpha48 or zsig97 expression is functionally relevant and may indicate a therapeutic target for the zalpha48 or zsig97, its agonists or antagonists. For example, a preferred transgenic mouse to engineer is one that over-expresses the zalpha48 or zsig97 mature polypeptide. Transgenic mice have been produced that overexpress zalpha48. They exhibit a curled-up bent over body and seem unable to extend it naturally. They also rapidly move their back legs in a scratching movement. This has been interpreted as a neurological or energy dysfunction. Moreover, such over-expression may result in a phenotype that shows similarity with human diseases. Similarly, knockout zalpha48 or zsig97 mice can be used to determine where zalpha48 or zsig97 is absolutely required in vivo. The phenotype of knockout mice is predictive of the in vivo effects of that a zalpha48 or zsig97 antagonist, such as those described herein, may have. The human zalpha48 or zsig97 cDNA can be used to isolate murine zalpha48 or zsig97 mRNA, cDNA and genomic DNA, which are subsequently used to generate knockout mice. These transgenic and knockout mice may be employed to study the zalpha48 or zsig97 gene and the protein encoded thereby in an in vivo system, and can be used as in vivo models for corresponding human or animal diseases (such as those in commercially viable animal populations).

For pharmaceutical use, the proteins of the present invention are formulated for parenteral, particularly intravenous or subcutaneous, delivery according to conventional methods. Intravenous administration will be by bolus injection or infusion over a typical period of one to several hours. In general, pharmaceutical formulations will include a zalpha48 or zsig97 polypeptide in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton, Pa., 19th ed., 1995. Therapeutic doses will generally be in the range of 0.1 to 100 μg/kg of patient weight per day, preferably 0.5-20 mg/kg per day, with the exact dose determined by the clinician according to accepted standards, taking into account the nature and severity of the condition to be treated, patient traits, etc. Determination of dose is within the level of ordinary skill in the art. The proteins may be administered for acute treatment, over one week or less, often over a period of one to three days or may be used in chronic treatment, over several months or years.

The invention is further illustrated by the following non-limiting examples.

### EXAMPLES

### Example 1

### Tissue Distribution

A. Tissue Distribution of zalpha48 or zsig97 Using Northern Blot

Human Multiple Tissue Northern Blots (MTN I, MTN II, and MTN III; Clontech) are probed to determine the tissue distribution of human zalpha48 or zsig97 expression. A probe is amplified from a human breast tumor or brain derived Marathon™-ready cDNA library (Clontech). Oligonucleotide primers are designed based on the EST sequence or cDNA sequence. The Marathon™-ready cDNA library is prepared according to manufacturer's instructions (Marathon™ cDNA Amplification Kit; Clontech) using human retina poly A+ RNA (Clontech). The probe is amplified in a polymerase chain reaction under reaction conditions, for example, as follows: 1 cycle at 94° C. for 1 minute; 35 cycles of 94° C. for 30 seconds and 68° C. for 1 minute 30 seconds; followed by 1 cycle at 72° C. for 10 minutes; followed by a 4° C. soak. The resulting DNA fragment is electrophoresed on an approximately 2% low melt agarose gel (SEA PLAQUE GTG low melt agarose, FMC Corp., Rockland, Me.), the fragment is purified using the QIAquick™ method (Qiagen, Chatsworth, Calif.), and the sequence is confirmed by sequence analysis.

The probe is radioactively labeled and purified as described herein using methods known in the art. ExpressHyb™ (Clontech) solution, or similar hybridization solution, is used for prehybridization and as a hybridizing solution for the Northern blots. Hybridization takes place overnight at 65° C. using about 1.0×10⁶ cpm/ml of labeled probe. The blots are then washed about 4 times at room temperature in 2×SSC, 0.05% SDS followed by about 2 washes at 50° C. in 0.1×SSC, 0.01% SDS for about 20 minutes each.

Additional analysis can be carried out on Northern blots made with poly(A) RNA from the human vascular cell lines HUVEC (human umbilical vein endothelial cells; Cascade Biologics, Inc., Portland, Oreg.), HPAEC (human pulmonary artery endothelial cells; Cascade Biologics, Inc.), HAEC (human aortic endothelial cells; Cascade Biologics, Inc.), AoSMC (aortic smooth muscle cells; Clonetics, San Diego, Calif.), UASMC (umbilical artery smooth muscle cells; Clonetics), HISM (human intestinal smooth muscle cells; ATCC CRL 7130), SK-5 (human dermal fibroblast cells; obtained from Dr. Russell Ross, University of Washington, Seattle, Wash.), NHLF (normal human lung fibroblast cells; Clonetics), and NHDF-NEO (normal human dermal fibroblast-neonatal cells; Clonetics). The probe is prepared and labeled and prehybridization and hybridization were carried out essentially as disclosed above. The blots are then washed at about 50° C. in 0.1×SSC, 0.05% SDS.

Additional analysis can be carried out on Northern blots made with poly(A) RNA from K-562 cells (erythroid, ATCC CCL 243), HUT78 cells (T cell, ATCC TIB-161), Jurkat cells (T cell), DAUDI (Burkitt's human lymphoma, Clontech, Palo Alto, Calif.), RAJI (Burkitt's human lymphoma, Clontech) and HL60 (Monocyte). The probe preparation and hybridization are carried out as above.

Additional analysis can be carried out on Northern blots made with poly (A) RNA from CD4⁺, CD8⁺, CD19⁺ and mixed lymphocyte reaction cells (CellPro, Bothell, Wash.) using probes and hybridization conditions described above. Additional analysis can be carried out on Human Brain Multiple Tissue Northern Blots II and III (Clontech) using the probe and hybridization conditions described above.

Moreover a Dot Blot is also performed using Human RNA Master Blots™ (Clontech). The methods and conditions for the Dot Blot were the same as for the Multiple Tissue Blots disclosed above. Again, a signal is present for those tissues that express the zalpha48 or zsig97 mRNA.

B. Tissue Distribution in Tissue Panels Using PCR

A panel of cDNAs from human tissues was screened for zalpha48 or zsig97 expression using PCR. The amplification was carried out as follows: 1 cycle at 94° C. for 2 minutes, 35 cycles of 94° C. for 30 seconds, 63.4° C. for 30 seconds and 72° C. for 30 seconds, followed by 1 cycle at 72° C. for 5 minutes. About 10 μl of the PCR reaction product was subjected to standard Agarose gel electrophoresis using a 4% agarose gel. For zalpha48 expression is seen in the pancreas, adrenal gland, ovary, and pituitary. For zsig97, expression is seen in the thyroid, prostate smooth muscle cell, prostate, KG-1 (dendritic cell line) and THP-1 (an acute monocytic leukemia cell line).

### Example 2

### Chromosomal Assignment and Placement of zalpha48 or zsig97

Zalpha48 or zsig97 is mapped to a human chromosome, such as chromosome 2 or 8, respectively, using the commercially available GeneBridge 4 Radiation Hybrid Panel (Research Genetics, Inc., Huntsville, Ala.). The GeneBridge 4 Radiation Hybrid Panel contains DNAs from each of 93 radiation hybrid clones, plus two control DNAs (the HFL donor and the A23 recipient). A publicly available WWW server (http://www-genome.wi.mit.edu/cgi-bin/contig/rhmapper.pl) allows mapping relative to the Whitehead Institute/MIT Center for Genome Research's radiation hybrid map of the human genome (the “WICGR” radiation hybrid map) which was constructed with the GeneBridge 4 Radiation Hybrid Panel.

For the mapping of zalpha48 or zsig97 with the GeneBridge 4 RH Panel, 20 μl reactions are set up in a 96-well microtiter plate (Stratagene, La Jolla, Calif.) and used in a RoboCycler Gradient 96 thermal cycler (Stratagene). Each of the 95 PCR reactions consist of 2 μl 10× KlenTaq PCR reaction buffer (Clontech), 1.6 μl dNTPs mix (2.5 mM each, PERKIN-ELMER, Foster City, Calif.), 1 μl sense primer, 1 μl antisense primer, 2 μl RediLoad (Research Genetics, Inc.), 0.4 μl 50× Advantage KlenTaq Polymerase Mix (Clontech), 25 ng of DNA from an individual hybrid clone or control and ddH₂O for a total volume of 20 μl. The reactions are overlaid with an equal amount of mineral oil and sealed. The PCR cycler conditions are, for example, as follows: an initial 1 cycle 5 minute denaturation at 95° C., 35 cycles of a 1 minute denaturation at 95° C., 1 minute annealing at 66° C. and 1.5 minute extension at 72° C., followed by a final 1 cycle extension of 7 minutes at 72° C. The reactions are separated by electrophoresis on a 2% agarose gel (Life Technologies, Gaithersburg, Md.).

An STS is defined by a pair of oligonucleotide primers that are used in a polymerase chain reaction, as describe above, to specifically detect this site in the presence of all other genomic sequences. Since STSs are based solely on DNA sequence they can be completely described within an electronic database, for example, Database of Sequence Tagged Sites (dbSTS), GenBank, (National Center for Biological Information, National Institutes of Health, Bethesda, Md. http://www.ncbi.nlm.nih.gov), and can be searched with a gene sequence of interest for the mapping data contained within these short genomic landmark STS sequences, or on the WICGR radiation hybrid map. Proximal and distal framework markers can be determined as well. The use of surrounding markers will position zalpha48 or zsig97 in a defined region on the integrated LDB chromosome map (The Genetic Location Database, University of Southhampton, WWW server: http://cedar. genetics.soton.ac.uk9/public_html/). These experiments placed zalpha48 at locus 2p25.3 and zsig97 at locus 8q11.23.

### Example 3

### Chemical Synthesis and Purification of Human Zalpha48 or zsig97 Peptides

Zalpha48 or zsig97 polypeptides, such as that shown in SEQ ID NO: 2 or 6, are synthesized by solid phase peptide synthesis using the ABI/PE Peptide Synthesizer model 431A (Applied Biosytems/Perkin Elmer (ABI/PE, Foster City, Calif.). One preferred zalpha48 or zsig97 peptide sequence is shown in SEQ ID NO:2 or 6. Other zalpha48 or zsig97 peptides include those described herein.

Fmoc-Amide resin is used for synthesis of the active zalpha48 or zsig97 peptide-amide and Fmoc-Asparagine resin are used for non-amidated zalpha48 or zsig97 peptides. The Fmoc-Amide resin (0.68 mmol/g) and the Fmoc-Asparagine resin (0.75 mmol/g) are purchased from ABI/PE. The amino acids can be purchased from AnaSpec, Inc., San Jose, Calif. in pre-weighed, 1 mmol cartridges. All the reagents except piperidine are purchased from ABI/PE. The piperidine is purchased from Aldrich, St. Louis Mo. Synthesis procedure is taken from the ABI Model 431A manual. Double coupling cycles are used during the high aggregation portion of the sequence, as predicted by Peptide Companion software (Peptides International, Louisville, Ky.).

The peptides are cleaved from the solid phase following the standard TFA cleavage procedure as outlined in the Peptide Cleavage protocol manual published by ABI/PE. Purification of the peptides is by RP-HPLC using a C18, 10 mm preparative column. Eluted fractions from the column are collected and analyzed for correct mass and purity by electrospray mass spectrometry. The analysis results should indicate that the Zalpha48 or zsig97 peptides are present and pure in one of the pools from the HPLC purification step. The pools containing each of the peptides is retained and lyophilized.

Post lyophilization, the Zalpha48 or zsig97 peptides are analyzed for purity using analytical HPLC. The analytical HPLC column used is a Vydac 10 cm, 5 um column. The analysis should result in 95% purity for Zalpha48 or zsig97 peptides. These peptides are prepared for use in subsequent biological assays.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1. An isolated polynucleotide encoding a polypeptide comprising a sequence of amino acid residues that is at least 90% identical to an amino acid sequence selected from the group consisting of an amino acid sequence as shown in SEQ ID NO: 2 from amino acid Asp 90 to amino acid Arg 133;
an amino acid sequence as shown in SEQ ID NO:2 from amino acid Asp 95 to amino acid Arg 133;
an amino acid sequence as shown in SEQ ID NO: 2 from amino acid Asp 90 to amino acid Lys 151;
and an amino acid sequence as shown in SEQ ID NO:2 from amino acid Asp 95 to amino acid Lys 151;
wherein amino acid substitutions in the isolated polypeptide consist of conservative amino acid substitutions.

2. An isolated polynucleotide encoding a polypeptide comprising a sequence of amino acid residues that is at least 90% identical to an amino acid sequence comprising selected from the group consisting of an amino acid sequence as shown in SEQ ID NO: 6 from amino acid Asp 69 to Arg 112;
an amino acid sequence as shown in SEQ ID NO:6 from amino acid Asp74 to amino acid Asp 112;
an amino acid sequence as shown in SEQ ID NO: 6 from amino acid Asp 69 to Thr 129;
and an amino acid sequence as shown in SEQ ID NO:6 from amino acid Asp74 to amino acid Thr 129;
wherein amino acid substitutions in the isolated polypeptide consist of conservative amino acid substitutions.

3. An isolated polynucleotide encoding a polypeptide comprising a sequence of amino acid residues that is at least 90% identical to an amino acid sequence comprising an amino acid sequence as shown in SEQ ID NO: 2 from amino acid Cys 134 to amino acid Lys 151
wherein amino acid substitutions in the isolated polypeptide consist of conservative amino acid substitutions.

4. An isolated polynucleotide encoding a polypeptide comprising a sequence of amino acid residues that is at least 90% identical to an amino acid sequence comprising an amino acid sequence as shown in SEQ ID NO: 6 from amino acid Cys 113 to amino acid Thr 129,
wherein amino acid substitutions in the isolated polypeptide consist of conservative amino acid substitutions.

5. An isolated polynucleotide sequence according to claim 1, wherein the polynucleotide comprises nucleotide 1 to nucleotide 1258 of SEQ ID NO:9.

6. An isolated polynucleotide sequence according to claim 2, wherein the polynucleotide comprises nucleotide 1 to nucleotide 814 of SEQ ID NO:10.

7. An isolated polynucleotide according to claim 1, wherein the polynucleotide encodes an amino acid sequence as shown in SEQ ID NO:2 from amino acid Gly 20 to amino acid Lys 151.

8. An isolated polynucleotide according to claim 2, wherein the polynucleotide encodes an amino acid sequence as shown in SEQ ID NO:6 from amino acid Ser 21 to amino acid Thr 129.

9. An expression vector comprising the following operably linked elements:
a transcription promoter;
a polynucleotide of claim 1 and
a transcription terminator.

10. An expression vector comprising the following operably linked elements:
a transcription promoter;
a polynucleotide of claim 2 and
a transcription terminator.

11. An expression vector according to claim 9, further comprising a secretory signal sequence operably linked to the DNA segment.

12. An expression vector according to claim 10, further comprising a secretory signal sequence operably linked to the DNA segment.

13. A cultured cell into which has been introduced an expression vector according to claim 11, wherein the cell expresses a polypeptide encoded by the DNA segment.

14. A cultured cell into which has been introduced an expression vector according to claim 12, wherein the cell expresses a polypeptide encoded by the DNA segment.

15. A DNA construct encoding a fusion protein, the DNA construct comprising:
a first DNA segment comprising a polynucleotide of claim 1 and
at least one other DNA segment encoding an additional polypeptide,
wherein the first and other DNA segments are connected in-frame; and
encode the fusion protein.

16. A DNA construct encoding a fusion protein, the DNA construct comprising:
a first DNA segment comprising a polynucleotide of claim 2 and
at least one other DNA segment encoding an additional polypeptide,
wherein the first and other DNA segments are connected in-frame; and
encode the fusion protein.

17. A fusion protein produced by a method comprising:
culturing a host cell into which has been introduced a vector comprising the following operably linked elements:
(a) a transcriptional promoter;
(b) a DNA construct encoding a fusion protein according to claim 15; and
(c) a transcriptional terminator; and
recovering the protein encoded by the DNA segment.

18. A fusion protein produced by a method comprising:
culturing a host cell into which has been introduced a vector comprising the following operably linked elements:
(a) a transcriptional promoter;
(b) a DNA construct encoding a fusion protein according to claim 16; and
(c) a transcriptional terminator; and
recovering the protein encoded by the DNA segment.

19. An isolated polypeptide comprising a sequence of amino acid residues encoded by a polypeptide of claim 1 wherein amino acid substitutions in the isolated polypeptide from the sequence encoded by the polypeptide of claim 1 consist of conservative amino acid substitutions.

20. An isolated polypeptide comprising a sequence of amino acid residues encoded by a polypeptide of claim 2 wherein amino acid substitutions in the isolated polypeptide from the sequence encoded by the polypeptide of claim 2 consist of conservative amino acid substitutions.

21. An isolated polypeptide comprising a sequence of amino acid residues that is at least 90% identical to an amino acid sequence comprising an amino acid sequence as shown in SEQ ID NO: 2 from polypeptide Cys 134 to polypeptide Lys 151
wherein amino acid substitutions in the isolated polypeptide consist of conservative amino acid substitutions.

22. An isolated polypeptide comprising a sequence of amino acid residues that is at least 90% identical to an amino acid sequence comprising an amino acid sequence as shown in SEQ ID NO: 6 from amino acid Cys 113 to amino acid Thr 129.
wherein amino acid substitutions in the isolated polypeptide consist of conservative amino acid substitutions.

23. A method of producing a polypeptide comprising:
culturing a cell according to claim 13; and isolating the polypeptide produced by the cell.

24. A method of producing a polypeptide comprising:
culturing a cell according to claim 14; and isolating the polypeptide produced by the cell.

25. A family of peptide hormones comprising a gene structure of four exons wherein two mature peptides are produced from the amino acids encoded by said exons; wherein said first exon comprises the nucleotides encoding the signal sequence, said second and third exons comprise the nucleotides encoding said first mature peptide and said fourth exon comprises the nucleotides encoding said second mature peptide.

26. A method of detecting, in a test sample, the presence of a modulator of zalpha48 protein activity, comprising:
transfecting a zalpha48-responsive cell, with a reporter gene construct that is responsive to a zalpha48-stimulated cellular pathway; and
producing a polypeptide by the method of claim 23; and
adding the polypeptide to the cell, in the presence and absence of a test sample; and
comparing levels of response to the polypeptide, in the presence and absence of the test sample, by a biological or biochemical assay; and
determining from the comparison, the presence of the modulator of zalpha48 activity in the test sample.

27. A method of detecting, in a test sample, the presence of a modulator of zsig97 protein activity, comprising:
transfecting a zsig97-responsive cell, with a reporter gene construct that is responsive to a zsig97-stimulated cellular pathway; and
producing a polypeptide by the method of claim 24; and
adding the polypeptide to the cell, in the presence and absence of a test sample; and
comparing levels of response to the polypeptide, in the presence and absence of the test sample, by a biological or biochemical assay; and
determining from the comparison, the presence of the modulator of zsig97 activity in the test sample.

28. A method of producing an antibody to a polypeptide comprising the steps of:
inoculating an animal with a polynucleotide of claim 1 or a polypeptide encoded by a polynucleotide of claim 1 wherein the polynucleotide or the polypeptide elicits an immune response in the animal to produce the antibody;
and isolating the antibody from the animal.

29. A method of producing an antibody to a polypeptide comprising the steps of:
inoculating an animal with a polynucleotide of claim 2 or a polypeptide encoded by a polynucleotide of claim 2 wherein the polynucleotide or the polypeptide elicits an immune response in the animal to produce the antibody;
and isolating the antibody from the animal.

30. An antibody produced by the method of claim 28, which binds to a polypeptide of SEQ ID NO:2.

31. An antibody produced by the method of claim 29, which binds to a polypeptide of SEQ ID NO:6.

32. The antibody of claim 30, wherein the antibody is a monoclonal antibody.

33. The antibody of claim 31, wherein the antibody is a monoclonal antibody.

34. A method for detecting pancreas, adrenal gland, ovary, or pituitary tissue in a patient sample, comprising:
obtaining a tissue or biological sample from a patient;
incubating the tissue or biological sample with an antibody of claim 30 under conditions wherein the antibody binds to its complementary polypeptide in the tissue or biological sample;
visualizing the antibody bound in the tissue or biological sample; and
comparing levels and localization of antibody bound in the tissue or biological sample from the patient to a non-pituitary, testis, or spleen control tissue or biological sample,
wherein an increase in the level or localization of antibody bound to the patient tissue or biological sample relative to the non-pancreas, adrenal gland, ovary, or pituitary control tissue or biological sample is indicative of pancreas, adrenal gland, ovary, or pituitary tissue in a patient sample.

35. A method for detecting thyroid or prostate tissue in a patient sample, comprising:
obtaining a tissue or biological sample from a patient;
incubating the tissue or biological sample with an antibody of claim 31 under conditions wherein the antibody binds to its complementary polypeptide in the tissue or biological sample;
visualizing the antibody bound in the tissue or biological sample; and
comparing levels and localization of antibody bound in the tissue or biological sample from the patient to a non-thyroid or prostate control tissue or biological sample,
wherein an increase in the level or localization of antibody bound to the patient tissue or biological sample relative to the non-thyroid or prostate control tissue or biological sample is indicative of thyroid or prostate tissue in a patient sample.

36. A method for detecting a pancreas, adrenal gland, ovary, or pituitary cancer in a patient, comprising:
obtaining a tissue or biological sample from a patient;
incubating the tissue or biological sample with an antibody of claim 30 under conditions wherein the antibody binds to its complementary polypeptide in the tissue or biological sample;
visualizing the antibody bound in the tissue or biological sample; and
comparing levels of antibody bound in the tissue or biological sample from the patient to a normal control tissue or biological sample,
wherein an increase in the level of antibody bound to the patient tissue or biological sample relative to the normal control tissue or biological sample is indicative of a pancreas, adrenal gland, ovary, or pituitary cancer in the patient.

37. A method for detecting a thyroid or prostate cancer in a patient, comprising:
obtaining a tissue or biological sample from a patient;
incubating the tissue or biological sample with an antibody of claim 31 under conditions wherein the antibody binds to its complementary polypeptide in the tissue or biological sample;
visualizing the antibody bound in the tissue or biological sample; and
comparing levels of antibody bound in the tissue or biological sample from the patient to a normal control tissue or biological sample,
wherein an increase in the level of antibody bound to the patient tissue or biological sample relative to the normal control tissue or biological sample is indicative of a pancreas, adrenal gland, ovary, or pituitary cancer in the patient.

38. A method for detecting pancreas, adrenal gland, ovary or pituitary tissue in a patient sample, comprising:
obtaining a tissue or biological sample from a patient;
labeling a polynucleotide comprising at least 14 contiguous nucleotides of a polypeptide of claim 1;
incubating the tissue or biological sample with under conditions wherein the polynucleotide will hybridize to complementary polynucleotide sequence;
visualizing the labeled polynucleotide in the tissue or biological sample; and
comparing the level and localization of labeled polynucleotide hybridization in the tissue or biological sample from the patient to a control non-pancreas, adrenal gland, ovary or pituitary tissue or biological sample,
wherein an increase in the level or localization of the labeled polynucleotide hybridization to the patient tissue or biological sample relative to the control non-pancreas, adrenal gland, ovary or pituitary tissue or biological sample is indicative of pancreas, adrenal gland, ovary or pituitary tissue in a patient sample.

39. A method for detecting thyroid or prostate tissue in a patient sample, comprising:
obtaining a tissue or biological sample from a patient;
labeling a polynucleotide comprising at least 14 contiguous nucleotides of a polypeptide of claim 2;
incubating the tissue or biological sample with under conditions wherein the polynucleotide will hybridize to complementary polynucleotide sequence;
visualizing the labeled polynucleotide in the tissue or biological sample; and
comparing the level and localization of labeled polynucleotide hybridization in the tissue or biological sample from the patient to a control non-thyroid or prostate tissue or biological sample,
wherein an increase in the level or localization of the labeled polynucleotide hybridization to the patient tissue or biological sample relative to the control non-thyroid or prostate tissue or biological sample is indicative of thyroid or prostate tissue in a patient sample.

40. A method for detecting a pancreas, adrenal gland, ovary, or pituitary cancer in a patient, comprising:
obtaining a tissue or biological sample from a patient;
labeling a polynucleotide comprising at least 14 contiguous nucleotides of a polypeptide of claim 1;
incubating the tissue or biological sample with under conditions wherein the polynucleotide will hybridize to complementary polynucleotide sequence;
visualizing the labeled polynucleotide in the tissue or biological sample; and
comparing the level of labeled polynucleotide hybridization in the tissue or biological sample from the patient to a normal control tissue or biological sample,
wherein an increase in the labeled polynucleotide hybridization to the patient tissue or biological sample relative to the normal control tissue or biological sample is indicative of a pancreas, adrenal gland, ovary, or pituitary cancer in the patient.

41. A method for detecting a thyroid or prostate cancer in a patient, comprising:
obtaining a tissue or biological sample from a patient;
labeling a polynucleotide comprising at least 14 contiguous nucleotides of a polypeptide of claim 2;
incubating the tissue or biological sample with under conditions wherein the polynucleotide will hybridize to complementary polynucleotide sequence;
visualizing the labeled polynucleotide in the tissue or biological sample; and
comparing the level of labeled polynucleotide hybridization in the tissue or biological sample from the patient to a normal control tissue or biological sample,
wherein an increase in the labeled polynucleotide hybridization to the patient tissue or biological sample relative to the normal control tissue or biological sample is indicative of a thyroid or prostate cancer in the patient.


SEQUENCE LISTING (10 sequences)

SEQ ID NO: 1 - DNA, 1258 bp, homo sapiens
gaattcggcacgaggcggggtccgcggaggtgaggggccgggtccagcgccagcggctcctcccgcctcccctcccctcccccgccccgcgctccgtccccctcccccgctgactttctctccggccccccgcgccccttctctcgcagcgagcccagctctcggcgcgtgtcggagtctcccagccccgcggccccgagcgcacgatgcgcggacccgggcaccccctcctcctggggctgctgctggtgctgggggcggcggggcgcggccgggggggcgcggagccccgggagccggcggacggacaggcgctgctgcggctggtggtggaactcgtccaggagctgcggaagcaccactcggcggagcacaagggcctgcagctcctcgggcgggactgcgccctgggccgcgcggaggcggcggggctggggccttcgccggagcagcgagtggaaattgttcctcgagatctgaggatgaaggacaagtttctaaaacaccttacaggccctctttattttagtccaaagtgcagcaaacacttccatagactttatcacaacaccagagactgcaccattcctgcatactataaaagatgcgccaggcttcttacccggctggctgtcagtccagtgtgcatggaggataagtgagcagaccgtacaggagcagcacaccaggagccatgagaagtgccttggaaaccaacagggaaacagaactatctttatacacatcccctcatggacaagagatttatttttgcagacagactcttccataagtcctttgagttttgtatgttgttgacagtttgcagatatatattcgataaatcagtgtacttgacagtgttatctgtcacttatttaaaaaaaaaacacaaaaggaatgctccacatttgacgtgtagtgctataaaacacagaatatttcattgtcttcattaggtgaaatcgcaaaaaatatttctttagaaacataagcagaatcttaaagtatattttcatataacataatttgatattctgtattactttcactgttaaattctcagagtattatttggaacggcatgaaaaattaaaatttcggtcatgttttagagacagtggagtgtaaatctgtggctaattctgttggtcgtttgtattataaatgtaaaatagtattccagctattgtgcaatatgtaaatagtgtaaataaacacaagtaataaaaaaaaaaaaaaaaaaatagcggccgcc

SEQ ID NO: 2 - protein, 151 aa, homo sapiens
MRGPGHPLLLGLLLVLGAAGRGRGGAEPREPADGQALLRLVVELVQELRKHHSAEHKGLQLLGRDCALGRAEAAGLGPSPEQRVEIVPRDLRMKDKFLKHLTGPLYFSPKCSKHFHRLYHNTRDCTIPAYYKRCARLLTRLAVSPVCMEDK

SEQ ID NO: 3 - DNA, 1508 bp, mus musculus
gtcgacccacgcgtccgcccacgcgtccgcccacgcgtccggtggggctggagctcatcaagtagtgtcgggactgggggaccgaggagcgcggcagacgcgggacctctggtgatcaggtgcccgaggacgacgcgggatccacggcgtgggcgcaggtgcccgagggcagtgcggggcccaccgctggcaggtgcccggacctctcaagtccagggctgcccatctgctccgcggctccggacctcggcagcccccggggcgtcacctgtggcctcgcatctcaaagaggtgtccaaagaccccgcgcgccgtccccgctgtgaaagaaagttgtgcggcgggctcaggctgcgcctggactccgcgtgcgggaaacttgggcgccggctgctggggcggcgggtcccaagacccgcgacacaggccaccagggccatgcgcgtgtccgggcgcccgatgctccttgcgctgctgctactgctgagcacggtgggggaccggggacgagcgcagtccagggggcctgcagaccggcaaacgctgctgaggttgctagttgagctggtccaggagctgaagaaattccacataggagactccaagaggttgcagctccttggcgagtccgactttgccctgggccgcagagaggccacggattatggggcagaccaagaggagcagagagtggagattgttcctcgagatctaaggatgaaggacaagtttctgaaacatctcacaggtcctctttatttcagtccgaagtgcagcaagcactttcacagactttaccacaacacgagagactgcaccatccccgcatactataaaagatgtgccaggttacttactcggctggcggtcagtcccatgtgcatggagagataagctgcaaaggagcgacacaccaggaaccttgaaaagtgccttggaaaccaacagaacagaaccatcttcatgcccacttcctgtggacagagatttatttttgcactcttccataattcctttgcattttctatgttgctgacagtttacaaatatgcattcagtgaatcagtgtatctgtcagtgagatttgccactcttatgattgtaaaaacagacaacattccacatttgatgtattgttctgtaaaatgaggtattttctttttaagattaggtggaaccacaaggaatatttctttagaaacataggcagaacattaaactgtattttcatagagcatatagtatgatttgacattttgtattctacaattaaaatcccaaagtatagtttggaactgcatgaaaaattttaatgaaaaccaaaaaaacctcagttgtgttgaaaacagtggcatatgtatctgtggctatgccaaggccagtcccacttgtcctctgtaaatgtaaaagtacattccaggtattgtacaatatgtaaatactgtgaataaatacaaggaaataaatgaagtgtttggaataataaaaaaaaaaaaaaagggcggccg

SEQ ID NO: 4 - protein, 150 aa, Mus musculus
MRVSGRPMLLALLLLLSTVGDRGRAQSRGPADRQTLLRLLVELVQELKKFHIGDSKRLQLLGESDFALGRREATDYGADQEEQRVEIVPRDLRMKDKFLKHLTGPLYFSPKCSKHFHRLYHNTRDCTIPAYYKRCARLLTRLAVSPMCME

SEQ ID NO: 5 - DNA, 814 bp, homo sapiens
cggcttgcgaacatgcggccccttaagcccggcgcccctttgcccgcactcttcctgctggcgctggctttgtccccgcacggagcccacgggaggccccgggggcgcaggggagcgcgcgtcacggataaggagcccaagccgttgcttttcctccccgcggccggggccggccggactcccagcggctcccggagcgcagaaatattcccaagagactctaacttaaaagacaaattcataaagcatttcacagggccggtcacattttcaccagaatgcagcaaacatttccaccgactctattacaataccagggagtgctcaacgccagcttattacaaaagatgtgctagattgttaacaagattagcagtgagtccactgtgctcccagacctagcaaaactaccctacatttcctaagaatgtacatctaatttgaagaaaaagtgcctcaaatcatgcaaaatgtaaaaaaagatgaaatttatatttttatggatattaagatgagtaaaataagagacttcccagaaataactggttagctgtttcctgtcatagaatggagtctttcttgctttatctttttgtgtatacagtaatttataattttgtaaaacagagtttgaatcgcatattgaaaattagatattaaaaattgtgtgattgtattttatttttactagatatattattttctttatatggttaacattctaattaaacatttaattgtgtaaattatatctgtgagtgccagtgagaaataatgatttttttgtatatgactgttagtatatatttgtcaa

SEQ ID NO: 6 - protein, 129 aa, homo sapiens
MRPLKPGAPLPALFLLALALSPHGAHGRPRGRRGARVTDKEPKPLLFLPAAGAGRTPSGSRSAEIFPRDSNLKDKFIKHFTGPVTFSPECSKHFHRLYYNTRECSTPAYYKRCARLLTRLAVSPLCSQT

SEQ ID NO: 7 - DNA, 381 bp, Mus musculus
atgtggctcactaagcccagcaccccggtgtccgcactgctgctgctggcgctggctttgtccccgcctgggactcaggggcggccccagagaagcttggcagcgcgtgtcgctgagctcaggccagagctcttcctccctgtgacagggactcgcctgcctcccagagcctcccggagcacagaaatattcccaagagacttgacattgaaagacaaattcataaagcatttcacagggcctgtcacattttcagctgaatgtagcaaacacttccatcgactttatcacaatacccgggattgctcgacgccagcttattataaaagatgtgctagattgctgacaagattagctgtgagtccactatgctcccagacc

SEQ ID NO: 8 - protein, 127 aa, Mus musculus
MWLTKPSTPVSALLLLALALSPPGTQGRPQRSLAARVAELRPELFLPVTGTRLPPRASRSTEIFPRDLTLKDKFIKHFTGPVTFSAECSKHFHRLYHNTRDCSTPAYYKRCARLLTRLAVSPLCSQT

SEQ ID NO: 9 - DNA, 453 bp, Artificial Sequence
  degenerate sequence
atgmgnggnccnggncayccnytnytnytnggnytnytnytngtnytnggngcngcnggnmgnggnmgnggnggngcngarccnmgngarccngcngayggncargcnytnytnmgnytngtngtngarytngtncargarytnmgnaarcaycaywsngcngarcayaarggnytncarytnytnggnmgngaytgygcnytnggnmgngcngargcngcnggnytnggnccnwsnccngarcarmgngtngarathgtnccnmgngayytnmgnatgaargayaarttyytnaarcayytnacnggnccnytntayttywsnccnaartgywsnaarcayttycaymgnytntaycayaayacnmgngaytgyacnathccngcntaytayaarmgntgygcnmgnytnytnacnmgnytngcngtnwsnccngtntgyatggargayaar

SEQ ID NO: 10 - DNA, 387 bp, Artificial Sequence
  degenerate sequence
atgmgnccnytnaarccnggngcnccnytnccngcnytnttyytnytngcnytngcnytnwsnccncayggngcncayggnmgnccnmgnggnmgnmgnggngcnmgngtnacngayaargarccnaarccnytnytnttyytnccngcngcnggngcnggnmgnacnccnwsnggnwsnmgnwsngcngarathttyccnmgngaywsnaayytnaargayaarttyathaarcayttyacnggnccngtnacnttywsnccngartgywsnaarcayttycaymgnytntaytayaayacnmgngartgywsnacnccngcntaytayaarmgntgygcnmgnytnytnacnmgnytngcngtnwsnccnytntgywsncaracn